(19) United States
(12) Patent Application Publication
(10) Pub. No.: US 2010/0166701 A1
(43) Pub. Date: Jul. 1, 2010

(54) METHOD FOR TREATING RENAL CELL CARCINOMA USING GLYCOGENOLYSIS INHIBITORS

(76) Inventor: Glendon John Parker, Salt Lake City, UT (US)

Correspondence Address: MORRISS OBRYANT COMPAGNI, P.C., 734 EAST 200 SOUTH, SALT LAKE CITY, UT, 84102, US

(21) Appl. No.: 12/394,038

(22) Filed: May 26, 2009

Related U.S. Application Data
(60) Provisional application No. 61/034,413, filed on Mar. 6, 2008.

(51) Int. Cl. A61K 38/20; A61K 31/404; A61K 38/21; A61K 31/70

(52) U.S. Cl. 424/85.2; 514/414; 424/85.7; 514/23

(57) ABSTRACT

Renal cell carcinoma (RCC) exhibits unpredictable behavior, high recurrence rate, insensitivity to Positron-Emission Tomography (PET), and poor response to existing cancer treatments such as chemotherapy. Inhibition of glycogenolysis can restrict RCC tumor growth and increase its susceptibility to other treatments, in particular, those that compromise access to nutrient, such as anti-angiogenenic compounds. This can also increase uptake of glucose derivatives and thus increase sensitivity of PET. Reduction of glycogen stores within RCC through treatment with iron-salts can also reduce tumor growth rate through reducing activation of the hypoxia pathway. Thus, synergistic treatment of RCC with glycogenolysis inhibitors or inhibitors of glycogenosis, and/or in combination with anti-angiogenic compounds, can increase sensitivity of tumor detection, slow tumor formation and/or metastasis.

### CROSS-REFERENCE TO RELATED APPLICATIONS

This application claims the benefit of priority to U.S. Provisional Patent Application Ser. No. 61/034,413 filed on Mar. 6, 2008, the disclosure of which is hereby incorporated herein, in its entirety, by this reference.

### STATEMENT REGARDING FEDERALLY SPONSORED RESEARCH

This invention was made with government support under Grant DK43526 awarded by the National Institutes of Health. The government has certain rights in the invention.

### BACKGROUND OF THE INVENTION

1. Field of the Invention

The present invention relates generally to biotechnology, more particularly to the treatment of renal cancer carcinoma via metabolic inhibition. In particular, the present invention relates to methods of treating cancer using a glycogenolysis inhibitor alone, or in combination with, a chemotherapeutic compound.

2. State of the Art

The American Cancer Society estimates that 36,000 new cases of kidney cancer will be diagnosed in 2005 and that 12,600 will die of the disease. Renal Cell Carcinoma (RCC) accounts for 90% of all kidney cancer and represents 2 to 3% of all malignancies. One third of RCC presents as metastatic disease, with a five-year survival rate of less than 10%.

About 80% of RCC occurs as clear cell carcinoma. Clear-cell variants of RCC show a dramatic increase in glucose accumulation. Specifically, RCC has been reported to contain 780 μg glucose/mg protein (Mayer, D. and P. Bannasch, J. Cancer Res. Clin. Oncol 114(4):369-72 (1988)). A later study measured glycogen at 586±174 μg/mg protein, with a significantly lower level of 265±156 μg/mg protein in a higher grade (grade III) tumor (Steinberg, P et al. Lab. Invest. 67(4):506-11 (1992)). In contrast, glycogen levels from 1 to 50 μg/mg protein have been shown in normal kidney tissue (Steinberg, P et al. (1992), Mayer, D. and P. Bannasch (1988)). Unfortunately, RCC is not responsive to conventional cancer therapies, such as cytotoxic chemotherapy, hormonal therapy and radiation, and is only slightly responsive to immunotherapy (American Cancer Society, Cancer Reference Information. (2005); Motzer, R. J. and P. Russo J. Urol. 163(2):408-17 (2000); Vogelzang, N. J. and W. M. Stadler, Lancet 352(9141):1691-6 (1998); Kim, H. L. et al. J. Urol. 173(5):1496-501 (2005); Drucker, B. J. Cancer Treat. Rev. 31(7):536-45 (2005)).

Cancer cells have been shown to be subject to intense metabolic demands, while suffering from compromised access to nutrients and oxygen (Steinberg, P. et al. (1992)). Carcinogenesis results in a metabolic shift to aerobic glycolysis and reduction in oxidative metabolism resulting in a greater reliance on glucose metabolism (Steinberg, P et al. (1992)). Consistent with this shift to anaerobic metabolism, analysis of the transcriptional profiles and proteomic changes are associated with RCC pathogenesis show that both lactate dehydrogenase mRNA and protein levels are increased. (Steinberg, P et al., (1992); Mayer, D. and P. Bannasch J. Cancer Res. Clin. Oncol 114(4):369-72 (1988); Liou, L. S. et al. BMC Urol. 4:9 (2004), Takahashi, M. et al. Proc. Natl. Acad. Sci. USA, 98(17):9754-9 (2001); Boer, J. M. et al. Genome Res. 11(11):1861-70 (2001); Young, A. N. et al. Am. J. Pathol. 158(5):1639-51 (2001); Shi, T. et al. Mol. Carcinog. 40(1):47-61 (2004), Shi, T. et al. Mol. Carcinog. 40(1):47-61 (2004)). Furthermore, the enzymes involved in glycogen metabolism are up-regulated in RCC (Mayer, D. and P. Bannasch (1988); Boer, J. M. et al. (2001), Takashi, M. et al. Jpn. J. Cancer Res 80(10):975-80 (1989)).

The most effective and widely used treatment of metastatic RCC is immunotherapy with high doses of interleukin-2 (IL-2) and/or interferon-α. IL-2 stimulates many cytotoxic cells of the immune system, and amplifies any endogenous host response against the RCC. IL-2 treatment gives an average response rate of 14 to 20% with about a 3% increased survival rate over five years (Drucker, B. J. 31(7):536-45 (2005); Krown, S. E. Cancer 59(3 Suppl):647-51 (1987)). However, IL-2 induces vascular leak syndrome with peripheral edema, weight gain, ascites, and/or plural effusions and pulmonary edema. These toxic side-effects are dose-limiting and require administration to be tightly supervised in an inpatient setting (Siegel, J. P. and R. K. Puri J. Clin. Oncol. 9(4):694-704 (1991)).

Another potential RCC treatment is anti-angiogenic therapy via inhibitors of vascular endothelial growth factor (VEGF) receptors. In RCC, the pro-angiogenic production of VEGF has been demonstrated to be up-regulated, whereas anti-angiogenic proteins, such as kininogen, were shown to be highly down-regulated (Takahashi, M. et al. (2001); Higgins, J. P. et al. Am. J. Pathol. 162(3):925-32 (2003)). VEGF receptor antagonists, such as the humanized monoclonal antibody bevacizumab (Avastin), and inhibitors of VEGF receptor tyrosine kinase activity, such as SU-11248 (sunitinib, Sutent) are currently in phase II clinical trials on RCC patients. High dose bevacizumab had a 30% response with no difference in survival after three years (Yang, J. C. et al. N Engl. J. Med. 349(5):427-34 (2003)). Clinical trials with sunitinib malate resulted in a response of 24% to 40%, with stable disease in an additional 27% to 46% of patients and a median increase in survival of 16.4 months (Motzer, R. J. et al. J. Clin. Oncol. 24(1):16-24 (2006)).

Such anti-angiogenic treatments reduce nutrient and oxygen delivery to cancer cells, especially in areas of new tumor growth resulting in nutrient deprivation and eventually necrosis (Yang, J. C. et al. (2003)).

The dependence of carcinoma on glucose availability, particularly after treatment with anti-angiogenic compounds is one underlying concept to the invention. The second and novel concept is targeting the potential role of glycogen metabolism, which regulates intracellular glucose availability. High levels of glycogen, such as those present in RCC cells, provide a ready source of glucose to fuel the metabolic demands of the cancer cell. Thus, intracellular glycogen stores could buffer the effects anti-angiogenic treatments thereby protecting the viability of RCC cells. Inhibition of glycogen metabolism therefore will be predicted to also restrict carcinoma metabolism, particularly in conjunction with agents restricting nutrient delivery to carcinoma cells, such as anti-angiogenic compounds (FIG. 1).

In diabetes, hepatic glucose output is increased and contributes to elevated blood glucose levels. Since glycogenolysis is a major contributor to hepatic glucose output, several pharmaceutical companies have successfully examined ways to inhibit liver glycogen phosphorylase. Glycogen phosphorylase contains a newly described indole-binding site (Hoover, D. J. et al. J. Med. Chem. 41(16):2934-8 (1998)). Glycogen phosphorylase inhibitors (GPIs) have been developed to specifically bind at the indole site and inhibit both liver and muscle isoforms of the enzyme with high affinity. These compounds have been shown to inhibit cancer cell proliferation and induce apoptosis in vitro (Lee, W. N. et al. Br. J. Cancer 91(12):2094-100 (2004); Schnier, J. B. et al. BMC Urol. 5(1):6 (2005)).

Metabolic analysis of cellular metabolism in pancreatic tumor cells revealed dramatic changes in carbohydrate metabolism. Flux into fatty acid synthesis and the pentose phosphate cycle was inhibited at GPI (CP-320626) concentrations below those required to see the anti-proliferative and pro-apoptotic effects. Flux of glucose through the TCA cycle was also decreased (Lee, W. N. et al. (2004)). Treatment of prostate cancer cells with the GPI CP-91149 resulted in a two-fold increase in glycogen content, and a 50% reduction in cell number (Schnier, J. B. et al. (2005)).

Clinical trials, as well as in vivo studies with diabetic Ob/Ob^{-/-} mice, have demonstrated that the drug is effective at reducing hyperglycemia without inducing hypoglycemia, has stable pharmacodynamics, can be administered orally, and is non toxic (Ercan-Fang, N. et al. Am. J. Physiol. Endocrinol. Metab. 289(3):E366-72 (2005)). Unfortunately, the efficacy of this class of drug in treating hyperglycemia fades after a period of four to eight weeks (J. Treadway, Pfizer Inc., personal communication). Gluconeogenesis may be up-regulated to compensate for the relatively reduced hepatic glucose output. It is also likely that lysosomal glycogenolysis will be increased to compensate for the inhibition of cytosolic glycogen phosphorylase activity.

1-Deoxynojirimycin (DNJ) and its derivatives effectively inhibit both lysosomal and cytoplasmic glycogenolysis by potently inhibiting both lysosomal α-glucosidase and the α1, 6-glucosidase activity of glycogen debranching enzyme (Andersson, U. et al. Biochem. Pharmacol. 67(4):697-705 (2004); Bollen, M. and W. Stalmans Eur. J. Biochem. 181(3):775-80 (1989)). However, many enzymes, particularly in the protein secretory pathway, share glucosidase activities. In deed the compound was developed as luminal activity in the endoplasmic reticulum is anti-viral (Branza-Nichita, N. et al. J. Virol. 75(8):3527-36 (2001)). In addition, N-alkylated-DNJ compounds inhibit ceramide-specific glucosyltransferase, which is involved in glycosphingolipid synthesis (Mellor, H. R. et al. Biochem. J. 381(Pt 3):861-6 (2004); Branza-Nichita, N. et al. J. Virol. 75(8):3527-36 (2001)). A level of DNJ specificity can be achieved by using different N-alkylated side-chains. N-nonyl-DNJ (NN-DNJ) treated mice had sustained increases in hepatic glycogen levels for at least 120 days. NN-DNJ treated mice did not lose body weight, did not suffer hypoglycemia below normal fasting levels, and had slightly lower serum non-esterified fatty acid and triglyceride levels (Andersson, U. et al. (2004)). DNJ derivatives a

The anti-viral and potential anti-diabetic effects of glycogenolysis inhibitors dominate the intellectual property associated with this class of compounds. Some cancers are caused by viruses, and there is an established link between diabetes and cancer, although no mechanism of the link is established. Given the prevalence and profile of these diseases and carcinoma it is not surprising that a general search of the USPTO patent database would result in a significant number of hits (the terms “glycogen” and “cancer” result in retrieving 3076 patents since 1976). A distinct majority of these patents are due to incidental co-incidence, such as incorporation of the term into broader terms, such as “cancer pain”, “glycogen synthase-kinase” (eg. #5827856, #6066632, #7279576, and #7427400), are included in the names of references (#7470542), or as part of an auxiliary technique (#6962788). Some combinations are due to potential side-effects, such as glycogenosis from the anti-viral activity of deoxynojirimycin (#7528153) or treatment of carcinoma with advanced glycation end-products (#7071298). Other combination are due to use of the proteins of glycogen metabolism without reference to glycogen metabolism itself, such as the use of glycogen phosphorylase as a marker for tissue damage (#7527980) or carcinoma (#7510710). Sometimes the link between glycogenolysis inhibitors and cancer treatment is due to the specific mode of action proposed in the patent but is secondary to the proposed mode of activity, such as anti-diabetic activity (as there is a undefined link between diabetes and cancer; #6897019) or anti-psoriatic activity and skin cancer (#5925376). Finally, some patents explicitly mention a role for inhibitors of glycogenolysis as potential therapies for carcinoma, either in isolation or in combination with other agents (#7425550, #7390814, #7226942, #7223786, #7214704, #7057046). These patents, all from the same source, are focused on the anti-diabetic effects of glycogen-phosphorylase inhibitor activities. Without exception the use of these compounds is in a list of “other maladies” including anti-hypertensive and anti-HIV therapies in an attempt to broaden the scope of their intellectual property. This application is unique in that there is a central, direct, and mechanistic, link between glycogenolytic inhibition and restriction of glucose availability to carcinoma cell metabolism. No direct link between these two concepts is apparent in either USPTO patent or the PubMed databases.

Since RCC exhibits unpredictable behavior, high recurrence rate and poor response to existing therapy, there is a need in the art to increase efficacy of existing therapies. As described, glycongenolysis inhibitors provide a means of slowing increased cellular metabolism in cancer cells. Inhibition of glycogenolysis may restrict RCC tumor growth and increase its susceptibility to other treatments, in particular, those that compromise access to nutrient, such as anti-angiogenesis compounds. Thus, synergistic treatment of RCC with glycogenolysis inhibitors and anti-angiogenic compounds may slow tumor formation and/or metastasis.

### BRIEF SUMMARY OF THE INVENTION

Disclosed herein is a method of treating a subject with cancer, the method comprising: administering to the individual an effective amount of at least one agent that inhibits glycogenolysis.

Also disclosed herein is a method of treating an individual having clear cell carcinoma, the method comprising: administering to the subject an effective amount of at least one agent that inhibits glycogenolysis in combination with at least one chemotherapeutic agent.

Further disclosed is a method of detecting cancer in a subject, the method comprising: administering to the subject an inhibitor of glycogenolysis; then administering to the subject a glucose-based composition; then monitoring uptake of the glucose-based composition by the cancer, thereby detecting the cancer

Also disclosed is a method of treating a subject with cancer, the method comprising: administering to the individual a compound inhibiting accumulation of glycogen removing the protection afforded by glycogenosis and activation of the hypoxia pathway, reducing tumor growth rate.

### BRIEF DESCRIPTION OF THE DRAWINGS

FIG. 1 is an illustration of the different sources of glucose available to a carcinoma cell.

FIG. 2A is a graph showing a comparison of glycogen concentration in RCC tissue and uninvolved tissue.

FIG. 2B is a graph showing a comparison of glycogen synthase (GS), glycogen phosphorylase (GP) and phosphorylated glycogen synthase (pGP) in RCC tissue an uninvolved tissue.

FIG. 2C is a picture of a gel showing the relative intensities of antibody in RCC tissue and uninvolved tissue measured by densitometry.

FIG. 3 is a graph showing a comparison of mitochondrial protein levels in RCC tissue and uninvolved tissue.

FIG. 4A is a graph of glycogen levels in response to different activators of the hypoxia pathway.

FIG. 4B is a graph showing a comparison of glycogen concentration in RCC tissue and uninvolved tissue.

FIG. 4C is an immunoblot of hypoxia inducible factor-1 levels in response to different activators of the hypoxia pathway.

FIG. 4D is a graph of glycogen levels in response to desferrioxamine activation of glycogenosis in the presence and absence of iron salts.

FIG. 5 shows DFO induced glycogenosis is dependent on HIF-1 protein.

FIG. 6 shows that glycogen reserves in RCC are integrated into carcinoma metabolism and that glycogenolysis can be specifically inhibited.

FIG. 7 shows glycogen accumulation protects CAKI-2 cells from nutrient deprivation-induced cell death (Example 3). This effect was due to glycogenosis as treatment with a specific glycogen phosphorylase inhibitor resulted in a loss of protection.

FIG. 8 shows that iron treatment reduces mouse renal cancer (Renca) tumor growth.

### DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### DEFINITIONS

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

As used herein, the terms “manage,” “managing” and “management” refer to the beneficial effects that a subject derives from administration of a prophylactic or therapeutic agent, which does not result in a cure of the disease or diseases. In certain embodiments, a subject is administered one or more prophylactic or therapeutic agents to “manage” a disease so as to prevent the progression or worsening of the disease or diseases.

As used herein, the terms “prevent”, “preventing” and “prevention” refer to the methods to avert or avoid a disease or disorder or delay the recurrence or onset of one or more symptoms of a disorder in a subject resulting from the administration of a prophylactic agent.

As used herein, the term “in combination” refers to the use of more than one prophylactic and/or therapeutic agents. The use of the term “in combination” does not restrict the order in which prophylactic and/or therapeutic agents are administered to a subject with a disorder, e.g., hyperproliferative cell disorder, especially cancer. A first prophylactic or therapeutic agent can be administered prior to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second prophylactic or therapeutic agent to a subject which had, has, or is susceptible to a disorder. The prophylactic or therapeutic agents are administered to a subject in a sequence and within a time interval such that the agent of the invention can act together with the other agent to provide an increased benefit than if they were administered otherwise. Any additional prophylactic or therapeutic agent can be administered in any order with the other additional prophylactic or therapeutic agents.

The term “suitable” as used herein refers to a group that is compatible with the compounds, products, or compositions as provided herein for the stated purpose. Suitability for the stated purpose may be determined by one of ordinary skill in the art using only routine experimentation.

As used herein, the terms “administer” when used to describe the dosage of a compound, means a single dose or multiple doses of the compound.

As used herein, “apoptosis” refers to a form of cell death that includes progressive contraction of cell volume with the preservation of the integrity of cytoplasmic organelles; condensation of chromatin (i.e., nuclear condensation), as viewed by light or electron microscopy; and/or DNA cleavage into nucleosome-sized fragments, as determined by centrifuged sedimentation assays. Cell death occurs when the membrane integrity of the cell is lost (e.g., membrane blebbing) with engulfment of intact cell fragments (“apoptotic bodies”) by phagocytic cells.

As used herein, the term “cancer treatment” means any treatment for cancer known in the art including, but not limited to, chemotherapy and radiation therapy.

As used herein, “tumor cells” means both cells derived from tumors, including malignant tumors, and cells immortalized in vitro. “Normal” cells refer to cells with normal growth characteristics that do not show abnormal proliferation.

As used herein the term “glycogenolysis-related cancers” refers to human cancer that utilizes glycogenolysis. Elevated concentration is determined by comparison to normal, non-cancerous tissues of a similar cell type. Renal cancer is an example of a cancer in which glycogenolysis takes place to the advantage of the tumor.

As used herein the term “glycogenosis” refers to the accumulation of glycogen to maximum or super-physiological limits.

As used herein, the terms “an individual identified as having cancer” and “cancer patient” are used interchangeably and are meant to refer to an individual who has been diagnosed as having cancer. There are numerous well known means for identifying an individual who has cancer. In some embodiments, a cancer diagnosis is made or confirmed using PET imaging. Some embodiments of the invention comprise the step of identifying individuals who have cancer.

As used herein, the term “cancer characterized by a high rate of aerobic glycolysis” refers to cancer having cells which exhibit a higher rate of aerobic glycolysis than those of the tissues surrounding it do. Such cancer cells take up above-average quantities of glucose from the environment. Cancer characterized by a high rate of aerobic glycolysis can be identified using PET imaging technology, along with fluorodeoxyglucose. The positive detection of a tumor using such a test indicates that the cancer is characterized by a high rate of aerobic glycolysis. PET methodologies are set forth in (Czernin, J. 2002 Acta Medica Austriaca 29:162-170), which is incorporated herein by reference.

As used herein, the term “therapeutically effective amount” is meant to refer to an amount of an active agent or combination of agents effective to ameliorate or prevent the symptoms, shrink tumor size, or prolong the survival of the patient being treated. Determination of a therapeutically effective amount is well within the capabilities of those skilled in the art, especially in light of the detailed disclosure provided herein.

“Therapy” and “to treat”, “treating” or “treatment” of disease includes any therapy or treatment that alleviates in any way the symptoms of a disease. These terms refer to any administration of a composition intended to alleviate the severity of a disorder, and includes treatment intended to cure the disease, provide relief from the symptoms of the disease and to prevent or arrest the development of the disease in an individual at risk from developing the disease or an individual having symptoms indicating the development of the disease in that individual.

As used herein the term “inhibit” or “inhibiting” refers to a statistically significant and measurable reduction in activity, preferably a reduction of at least about 10% versus control, more preferably a reduction of about 50% or more, still more preferably a reduction of about 80% or more.

The terms “antagonist” and “inhibitor” are used interchangeably to refer to an agent that decreases or suppresses a biological activity, such as to repress an activity of glycogenolysis.

An “effective amount” of, e.g., a glycogenolysis inhibitor, with respect to the subject method of treatment, refers to an amount of the inhibitor in a preparation which, when applied as part of a desired dosage regimen brings about a desired clinical or functional result. When a particular functional activity is only readily observable in an in vitro assay, the ability of a compound to inhibit glycogenolysis in that in vitro assay serves as a reasonable proxy for the activity of that compound.

The term “prevent” or “preventing” when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount. Prevention of an infection includes, for example, reducing the number of diagnoses of the infection in a treated population versus an untreated control population, and/or delaying the onset of symptoms of the infection in a treated population versus an untreated control population. Prevention of pain includes, for example, reducing the magnitude of, or alternatively delaying, pain sensations experienced by subjects in a treated population versus an untreated control population.

The present invention provides compounds which are in prodrug form. The term “prodrug” is intended to encompass compounds that, under physiological conditions, are converted into the therapeutically active agents of the present invention. A common method for making a prodrug is to include selected moieties that are hydrolyzed under physiological conditions to reveal the desired molecule. In other embodiments, the prodrug is converted by an enzymatic activity of the host animal. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

General

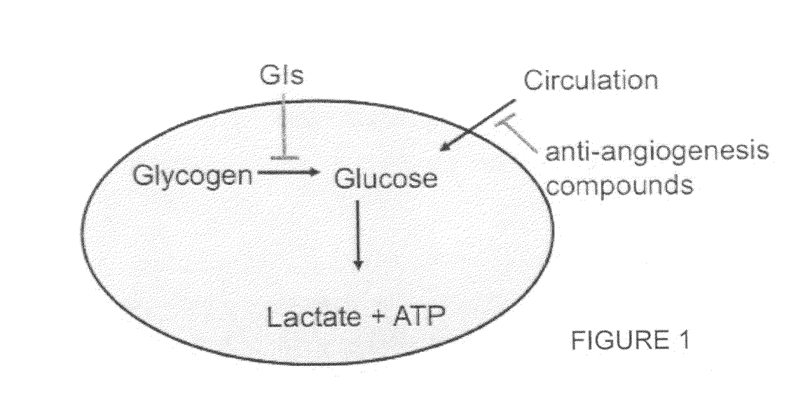

The metabolism of carcinomas is often distinctive in that they do not depend on the availability of oxygen. This restricts the metabolic options for the carcinoma and results in an increased dependence on anaerobic glucose metabolism. Many carcinoma types, particularly renal cell carcinoma, endometrial and thyroid carcinoma, also have super accumulations of glycogen, which is the mechanism by which cells store glucose reserves. Given the increased metabolic demands of the carcinoma, these accumulations can to be beneficial to cell survival. Restricting metabolic access to these reserves can increase the susceptibility of carcinoma to chemotherapies, particularly anti-angiogenic agents that restrict external nutrient availability. This includes both those carcinomas that have super accumulations of glycogen, as well as any other type of cancer (FIG. 1).

This concept has been verified by the inventor in an in vitro renal cell carcinoma model (Example 2 and 3). When these cells are subjected to an environment of restricted nutrient availability, they begin to undergo cell death. Prior accumulation of glycogen in these cells results in protection against cell death. However, this protection is lost when co-treated with an agent that prevents glycogen breakdown.

As demonstrated in Example 2 and 3, glycogenolysis inhibitors prevent access to internal reserves of cellular glucose. This increases the dependence on external nutrient availability and results in increased susceptibility to therapeutic agents that either increase the metabolic demands on the carcinoma cell, such as traditional chemotherapy agents, or reduce availability to external nutrient reserves, such as anti-angiogenic drugs.

Other non-glycogenotic carcinomas also have the potential to be treated by glycogenolysis inhibitors. Even in the absence of large intracellular glycogen reserves, glycogen metabolism is often coupled to other metabolic pathways, such as glycolysis and gluconeogenesis. A consequence of this observation is that inhibition of glycogenolysis can change other metabolic pathways even in the absence of large intracellular glycogen reserves.

The present invention includes a method for treating an individual with cancer. The method includes administering a glycogenolysis inhibitor in combination with a chemotherapeutic agent. Suitable glycogenolysis inhibitors inhibit the release of glucose from internal glycogen stores. For example, glycogen phosphorylase, which mediates the cytoplasmic release of glucose-1-phosphate, can be inhibited by CP-3616819. Glycogen breakdown in the lysosome can be inhibited by derivatives of deoxynojirimycin (DNJ) such as N-(n-Nonyl)deoxynojirimycin (NN-DNJ). Glycogenolysis inhibitors can remove metabolic access to internal reserves of glucose and thus, they may be synergistic with chemotherapeutic agents. For example, VEGF-receptor inhibitors are a class of chemotherapeutic agents that slow tumor growth and migration by inhibiting angiogenesis.

Treatment Methods

Methods described herein can be used to treat any disease or condition for which it is beneficial to inhibit glycogenolysis. One focus of the methods described herein is to achieve a therapeutic result by decreasing glycogenolysis via modulation of components of the glycogenolysis pathway. Thus, certain methods described herein can be used to treat any disease or condition susceptible to treatment by decreasing glycogenolysis.

In some embodiments, a disclosed method is applied to modulating glycogenolysis in vivo, in vitro, or ex vivo. In in vivo embodiments, the cells may be present in a tissue or organ of a subject animal or human being. In applications to an animal or human being, the disclosure includes a method of bringing cells into contact with a glycogenolysis inhibitor, optionally in combination with one or more other chemotherapeutic agents, in effective amounts to result in a decrease in glycogenolysis in comparison to the absence of the agent or combination. A non-limiting example is in the administration of the agent or combination to the animal or human being. Such contacting or administration may also be described as exogenously supplying the combination to a cell or tissue. Disclosed herein is a method of treating a subject with cancer, the method comprising administering to the subject an effective amount of at least one agent that inhibits glycogenolysis in combination with at least one chemotherapeutic agent.

Any form of cancer can be treated using this method, because of the synergistic effects with other chemotherapeutic compounds with the glycogenolysis inhibitors disclosed herein. For example, anti-angiogenic compounds can increase the dependence of the cancer cell on glycogen metabolism. As is shown in Example 3, glycogenolysis inhibitors can be used in combination with azide. An example of an anti-angiogenic compound is the VEGF-receptor, such as SU-11248. The chemotherapeutic agent can also be interleukin-2 or interferon-α.

The glycogenolysis inhibitor can inhibit at least one of glycogen phosphorylase, lysosomal α-glucosidase and phosphoglucomutase. One example of a glycogenolysis inhibitor is is N-(n-Nonyl)deoxynojirimycin. Another is CP-3616819. The glycogenolysis inhibitor can also decrease cellular metabolism, thereby increasing effectiveness of the chemotherapeutic agent.

Also disclosed is a method of treating an individual having clear cell carcinoma, the method comprising: administering to the individual an effective amount of at least one agent that inhibits glycogenolysis. Many carcinoma types, particularly renal cell carcinoma, endometrial and thyroid carcinoma, have super accumulations of glycogen, which is the mechanism by which cells store glucose reserves. Given the increased metabolic demands of the carcinoma, these accumulations can be beneficial to cell survival. Restricting metabolic access to these reserves can increase the susceptibility of carcinoma to chemotherapies, particularly anti-angiogenic agents that restrict external nutrient availability. This includes both those carcinomas that have super accumulations of glycogen, as well as any other type of cancer.

In particular embodiments, the methods and compositions of the invention comprise the administration of one or more glycogenolysis inhibitors (alone or in combination with other anti-cancer agents) to subjects/patients suffering from or expected to suffer from cancer, e.g., have a genetic predisposition for a particular type of cancer, have been exposed to a carcinogen, or are in remission from a particular cancer. The methods and compositions of the invention may be used as a first line or second line cancer treatment. Included in the invention is also the treatment of patients undergoing other cancer therapies and the methods and compositions of the invention can be used before any adverse effects or intolerance of these other cancer therapies occurs. The invention also encompasses methods for administering one or more compounds of the invention to treat or ameliorate symptoms in refractory patients. In a certain embodiment, that a cancer is refractory to a therapy means that at least some significant portion of the cancer cells are not killed or their cell division arrested. The determination of whether the cancer cells are refractory can be made either in vivo or in vitro by any method known in the art for assaying the effectiveness of treatment on cancer cells, using the art-accepted meanings of “refractory” in such a context. In various embodiments, a cancer is refractory where the number of cancer cells has not been significantly reduced, or has increased. Also disclosed are methods for administering one or glycogenolysis inhibitors to prevent the onset or recurrence of cancer in patients predisposed to having cancer.

In alternate embodiments, disclosed herein are methods for treating cancer in a subject by administering glycogenolysis inhibitors in combination with any other treatment or to patients who have proven refractory to other treatments but are no longer on these treatments. In certain embodiments, the patients being treated by the methods of the invention are patients already being treated with chemotherapy, radiation therapy, hormonal therapy, or biological therapy/immunotherapy. Among these patients are refractory patients and those with cancer despite treatment with existing cancer therapies. In other embodiments, the patients have been treated and have no disease activity and one or more glycogenolysis inhibitors of the invention are administered to prevent the recurrence of cancer.

Additionally, disclosed herein are methods of treatment of cancer as an alternative to chemotherapy, radiation therapy, hormonal therapy, and/or biological therapy/immunotherapy where the therapy has proven or may prove too toxic, i.e., results in unacceptable or unbearable side effects, for the subject being treated. The subject being treated with the methods of the invention may, optionally, be treated with other cancer treatments such as surgery, chemotherapy, radiation therapy, hormonal therapy or biological therapy, depending on which treatment was found to be unacceptable or unbearable.

Embodiments of a first aspect of the disclosure include a method of inhibiting or decreasing glycogenolysis by contacting one or more cells with a glycogenolysis inhibitor, optionally in combination with one or more other chemotherapeutic agents. The amount of a glycogenolysis inhibitor, or a combination thereof with one or more other chemotherapeutic agents, may be selected to be effective to produce an improvement in a treated subject, or a detectable reduction in glycogenolysis in vitro.

Monitoring and Diagnosing

Also disclosed herein is a method of diagnosing and monitoring renal cell carcinoma in an individual comprising: obtaining a sample from the individual; measuring in the sample a level of at least one protein involved in cellular metabolism; and determining a presence or an increased metastatic potential of clear cell carcinoma based on the level of the at least one protein involved in cellular metabolism. For example, the at least one protein can be an enzyme involved in anaerobic non-oxidative glucose metabolism. The enzyme can be involved in anaerobic non-oxidative glucose metabolism and be up-regulated, indicating the presence or the increased metastatic potential renal cell carcinoma. In another example, the at least one protein can be an enzyme involved in oxidative metabolism. The enzyme involved in oxidative metabolism can be down-regulated, indicating the presence or increased metastatic potential of renal cell carcinoma.

Some cancer types are not effective at visualization with positron emission tomography (PET scans). Renal cell carcinoma in particular does not always visualize. The basis for this imaging technique is the uptake of glucose into carcinoma cells, due to the profound changes in metabolism that occur as part of carcinogenesis. For example, renal cell carcinoma, which has ample glycogen reserves, may not need to import glucose and thus can be invisible in PET scans. Therefore, inhibition of glycogenolysis can increase glucose uptake in carcinoma. Therefore, glycogen-rich renal cell carcinomas, which may not take up enough fluorodeoxyglucose to be detected, can be detected when treated with a glycogenolysis inhibitor. It can also increase fluorodeoxyglucose uptake in all tumors, resulting in an overall increase in PET scan sensitivity. This can result in smaller tumors now being detected and medical intervention occurring at an earlier time point.

Therefore, disclosed herein is a method of detecting cancer in a subject, the method comprising: administering to the subject an inhibitor of glycogenolysis; then administering to the subject a glucose-based composition; then monitoring uptake of the glucose-based composition by the cancer, thereby detecting the cancer. For example, the test can be a PET scan, and the glucose-based composition can be fluorodeoxyglucose. In one example, administering an inhibitor of glycogenolysis can result in a 10, 20, 30, 40, 50, 60, 70, 80, or 90% or more increase uptake of the glucose-based composition by the cancer cells.

Also disclosed is a method of screening for a test compound that inhibits glycogenolysis, comprising: contacting components of the glycogenolysis pathway with a test compound; detecting interaction between a component of the pathway and the test compound; wherein interaction between the pathway component and the test compound indicates a test compound that inhibits glycogenolysis. The ability to modulate glycogenolysis can be measured by contacting the test compound with one or more tumor cells. This can be done in a high throughput assay system. The high throughput assay system can comprise an immobilized array of test compounds. Lastly, disclosed are compounds identified by these methods.

In a preferred specific embodiment, the invention encompasses a method for treatment, prevention and/or management of diseases or disorders associated with overexpression of glycogenolysis (e.g., cancer) comprising administering a therapeutically and/or prophylactically effective amount of an inhibitor as disclosed herein.

Identification of Subjects and Patients

The disclosure includes methods comprising identification of an individual suffering from one or more disease, disorders, or conditions, or a symptom thereof, and administering to the subject or patient a glycogenolysis inhibiting agent, optionally in combination with one or more other chemotherapeutic agents, as described herein. The identification of a subject or patient as having one or more diseases, disorders or conditions, or a symptom thereof, may be made by a skilled practitioner using any appropriate means known in the field. The disclosure also includes identification or diagnosis of a subject or patient as having one or more diseases, disorders or conditions, or a symptom thereof, which is suitably or beneficially treated or addressed by decreasing glycogenolysis in the subject or patient.

The subsequent administration of a glycogenolysis inhibitor, alone or in combination as described herein may be based on, or as directed by, the identification or diagnosis of a subject or patient as in need of one or more effects provided by a glycogenolysis inhibitor or a combination.

The disclosed compositions can be used to treat any disease where uncontrolled cellular proliferation occurs such as cancers. A non-limiting list of different types of cancers is as follows: lymphomas (Hodgkins and non-Hodgkins), leukemias, carcinomas, carcinomas of solid tissues, squamous cell carcinomas, adenocarcinomas, sarcomas, gliomas, high grade gliomas, blastomas, neuroblastomas, plasmacytomas, histiocytomas, melanomas, adenomas, hypoxic tumours, myelomas, AIDS-related lymphomas or sarcomas, metastatic cancers, or cancers in general.

A representative but non-limiting list of cancers that the disclosed compositions can be used to treat is the following: lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, colon cancer, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, or pancreatic cancer.

Compounds disclosed herein may also be used for the treatment of precancer conditions such as cervical and anal dysplasias, other dysplasias, severe dysplasias, hyperplasias, atypical hyperplasias, and neoplasias.

### Glycogenolysis Inhibitors

A “glycogenolysis inhibitor” of the disclosure is a ligand which modulates activity of glycogenolysis. In some cases, the ligand may bind or interact with a receptor in the glycogenolysis pathway. In other cases, the agent may modulate activity indirectly as described herein. In some embodiments, the agent is an agonist of one or more subtypes. In additional embodiments, the agent is an antagonist of glycogenolysis receptor activity.

Suitable glycogenolysis inhibitors can inhibit one or more of the steps involved in glycogenolysis including, but not limited to inhibiting glycogen phosphorylase (as discussed above), inhibiting the debranching enzyme that moves the remaining glucose units to another non-reducing end, and inhibiting the lysosomal conversion of glycogen to glucose through acid alpha-glucosidase.

A glycogenolysis inhibitor useful in a method described herein includes an agent that modulates glycogenolysis at the molecular level (e.g., by binding directly to the receptor), at the transcriptional and/or translational level (e.g., by preventing gene expression of glycogenolysis proteins), and/or by other modes (e.g., by binding to a substrate or co-factor of a receptor, or by modulating the activity of an agent that directly or indirectly modulates glycogenolysis activity). For example, in some embodiments, a glycogenolysis inhibitor is a compound that modulates the activity of an endogenous glycogenolysis receptor. The glycogenolysis inhibitor can be any, including, but not limited to, a chemical compound, a protein or polypeptide, a peptidomimetic, or an antisense molecule or ribozyme. A number of structurally diverse molecules with glycogenolysis modulating activity are known in the art. Structures, synthetic processes, safety profiles, biological activity data, methods for determining biological activity, pharmaceutical preparations, and methods of administration for a glycogenolysis inhibitor useful in a method described herein are described in the instant text and in the cited references, all of which are herein incorporated by reference in their entirety.

Examples of glycogenolysis inhibitors include, but are not limited to, chloroindole-carboxamides, imino-sugars, nojirimycin derivatives, purine nucleoside derivatives, glucopyranosylamine analogs and dihydropyridine derivatives.

A glycogenolysis inhibitor as described herein includes pharmaceutically acceptable salts, derivatives, prodrugs, metabolites, stereoisomer, or other variant of the agent. In some embodiments, a glycogenolysis inhibitor is chemically modified to reduce side effects, toxicity, solubility, and/or other characteristics. Methods for preparing and administering salts, derivatives, prodrugs, and metabolites of various compounds are well known in the art.

In some embodiments, a glycogenolysis inhibitor is an antisense nucleotide (e.g., siRNA) that specifically hybridizes with the cellular mRNA and/or genomic DNA corresponding to the gene(s) of a target receptor, or a molecule that otherwise modulates glycogenolysis, so as to inhibit their transcription and/or translation, or a ribozyme that specifically cleaves the mRNA of a target protein. Antisense nucleotides and ribozymes can be delivered directly to cells, or indirectly via an expression vector which produces the nucleotide when transcribed in the cell. Methods for designing and administering antisense oligonucleotides and ribozymes are known in the art, and are described, e.g., in Mautino et al., Hum Gene Ther 13:1027-37 (2002) and Pachori et al., Hypertension 39:969-75 (2002), herein incorporated by reference. In some embodiments, glycogenolysis modulation is achieved by administering a combination of at least one receptor modulator, and at least one transcriptional/translational modulator.

Compounds described herein that contain a chiral center include all possible stereoisomers of the compound, including compositions comprising the racemic mixture of the two enantiomers, as well as compositions comprising each enantiomer individually, substantially free of the other enantiomer. Thus, for example, contemplated herein is a composition comprising the S enantiomer substantially free of the R enantiomer, or the R enantiomer substantially free of the S enantiomer. If the named compound comprises more than one chiral center, the scope of the present disclosure also includes compositions comprising mixtures of varying proportions between the diastereomers, as well as compositions comprising one or more diastereomers substantially free of one or more of the other diastereomers. By “substantially free” it is meant that the composition comprises less than 25%, 15%, 10%, 8%, 5%, 3%, or less than 1% of the minor enantiomer or diastereomer(s). Methods for synthesizing, isolating, preparing, and administering various stereoisomers are known in the art.

In some preferred embodiments, compositions comprising one or more stereoisomers substantially free from one or more other stereoisomers provide enhanced affinity, potency, selectivity and/or therapeutic efficacy relative to compositions comprising a greater proportion of the minor stereoisomer(s). For example, the R-(−)-enantiomer of baclofen is about 100 times more active than the S-(+)-enantiomer against GABA-B receptors. Additional GABA modulators with stereoselective activities, and methods for separating and/or synthesizing particular stereoisomers, are known in the art, and described, e.g., in Zhu et al., J Chromatogr B Analyt Technol Biomed Life Sci., 785(2):277-83 (2003), Ansar et al., Therapie, 54(5):651-8 (1999), Karla et al., J Med. Chem., 42(11):2053-9 (1992), Castelli et al., Eur J. Pharmacol., 446(1-3):1-5 (2002), and Doyle et al., Chirality, 14(2-3):169-72 (2002).

As described herein, a glycogenolysis inhibitor, optionally in combination with one or more other chemotherapeutic agents, is administered to an animal or human subject to result in a decrease in glycogenolysis. A combination may thus be used to treat a disease, disorder, or condition of the disclosure.

### Combination Therapy

In some embodiments, a combination is of a glycogenolysis inhibitor administered with another chemotherapeutic agent. In some embodiments, the additional chemotherapeutic modulating agent modulates one or more aspects of cancer, e.g., proliferation, differentiation, migration and/or survival.

Prophylactic and therapeutic compounds that may be used in the methods and compositions of the invention include, but are not limited to, proteinaceous molecules, including, but not limited to, peptides, polypeptides, proteins, including post-translationally modified proteins, antibodies, etc.; small molecules (less than 1000 daltons), inorganic or organic compounds; nucleic acid molecules including, but not limited to, double-stranded or single-stranded DNA, double-stranded or single-stranded RNA, as well as triple helix nucleic acid molecules. Prophylactic and therapeutic compounds can be derived from any known organism (including, but not limited to, animals, plants, bacteria, fungi, and protista, or viruses) or from a library of synthetic molecules. In certain embodiments, one or more compounds of the invention are administered to a mammal, preferably a human, concurrently with one or more other therapeutic agents useful for the treatment of cancer or a disorder. The term “concurrently” is not limited to the administration of prophylactic or therapeutic agents at exactly the same time, but rather it is meant that compounds of the invention and the other agent are administered to a subject in a sequence and within a time interval such that the compounds of the invention can act together with the other agent to provide an increased benefit than if they were administered otherwise. For example, each prophylactic or therapeutic agent may be administered at the same time or sequentially in any order at different points in time; however, if not administered at the same time, they should be administered sufficiently close in time so as to provide the desired therapeutic or prophylactic effect. Each therapeutic agent can be administered separately, in any appropriate form and by any suitable route.

In various embodiments, the prophylactic or therapeutic agents are administered less than 1 hour apart, at about 1 hour apart, at about 1 hour to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, no more than 24 hours apart or no more than 48 hours apart. In preferred embodiments, two or more components are administered within the same patient visit.

A “chemotherapeutic agent” or “chemotherapeutic compound” is a chemical compound useful in the treatment of cancer. Chemotherapeutic cancer agents that can be used in combination with those disclosed herein include, but are not limited to, mitotic inhibitors (vinca alkaloids). These include vincristine, vinblastine, vindesine and Navelbine™ (vinorelbine,5′-noranhydroblastine). In yet other embodiments, chemotherapeutic cancer agents include topoisomerase I inhibitors, such as camptothecin compounds. As used herein, “camptothecin compounds” include Camptosar™ (irinotecan HCL), Hycamtin™ (topotecan HCL) and other compounds derived from camptothecin and its analogues. Another category of chemotherapeutic cancer agents that may be used in the methods and compositions of the invention are podophyllotoxin derivatives, such as etoposide, teniposide and mitopodozide. The invention further encompasses other chemotherapeutic cancer agents known as alkylating agents, which alkylate the genetic material in tumor cells. These include without limitation cisplatin, cyclophosphamide, nitrogen mustard, trimethylene thiophosphoramide, carmustine, busulfan, chlorambucil, belustine, uracil mustard, chlomaphazin, and dacarbazine. The invention encompasses antimetabolites as chemotherapeutic agents. Examples of these types of agents include cytosine arabinoside, fluorouracil, methotrexate, mercaptopurine, azathioprime, and procarbazine. An additional category of chemotherapeutic cancer agents that may be used in the methods and compositions of the invention include antibiotics. Examples include without limitation doxorubicin, bleomycin, dactinomycin, daunorubicin, mithramycin, mitomycin, mytomycin C, and daunomycin. There are numerous liposomal formulations commercially available for these compounds. The invention further encompasses other chemotherapeutic cancer agents including without limitation anti-tumor antibodies, dacarbazine, azacytidine, amsacrine, melphalan, ifosfamide and mitoxantrone.

The compositions disclosed herein can be administered alone or in combination with other anti-tumor agents, including cytotoxic/antineoplastic agents and anti-angiogenic agents. Cytotoxic/anti-neoplastic agents are defined as agents which attack and kill cancer cells. Some cytotoxic/anti-neoplastic agents are alkylating agents, which alkylate the genetic material in tumor cells, e.g., cis-platin, cyclophosphamide, nitrogen mustard, trimethylene thiophosphoramide, carmustine, busulfan, chlorambucil, belustine, uracil mustard, chlomaphazin, and dacabazine. Other cytotoxic/anti-neoplastic agents are antimetabolites for tumor cells, e.g., cytosine arabinoside, fluorouracil, methotrexate, mercaptopuirine, azathioprime, and procarbazine. Other cytotoxic/anti-neoplastic agents are antibiotics, e.g., doxorubicin, bleomycin, dactinomycin, daunorubicin, mithramycin, mitomycin, mytomycin C, and daunomycin. There are numerous liposomal formulations commercially available for these compounds. Still other cytotoxic/anti-neoplastic agents are mitotic inhibitors (vinca alkaloids). These include vincristine, vinblastine and etoposide. Miscellaneous cytotoxic/anti-neoplastic agents include taxol and its derivatives, L-asparaginase, anti-tumor antibodies, dacarbazine, azacytidine, amsacrine, melphalan, VM-26, ifosfamide, mitoxantrone, and vindesine.

Anti-angiogenic agents are well known to those of skill in the art. Suitable anti-angiogenic agents for use in the methods and compositions of the invention include anti-VEGF antibodies, including humanized and chimeric antibodies, anti-VEGF aptamers and antisense oligonucleotides. Other known inhibitors of angiogenesis include angiostatin, endostatin, interferons, interleukin 1 (including α and β) interleukin 12, retinoic acid, and tissue inhibitors of metalloproteinase-1 and -2. (TIMP-1 and -2). Small molecules, including topoisomerases such as razoxane, a topoisomerase II inhibitor with anti-angiogenic activity, can also be used.

Other anti-cancer agents that can be used in combination with glycogenolysis inhibitors, including pharmaceutical compositions and dosage forms and kits of the invention, include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride. Other anti-cancer drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminol evulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer. Preferred additional anti-cancer drugs are 5-fluorouracil and leucovorin.

### Formulations and Doses

In some embodiments of the disclosure, a glycogenolysis inhibitor, optionally in combination with one or more other chemotherapeutic agents, is in the form of a composition that includes at least one pharmaceutically acceptable excipient. As used herein, the term “pharmaceutically acceptable excipient” includes any excipient known in the field as suitable for pharmaceutical application. Suitable pharmaceutical excipients and formulations are known in the art and are described, for example, in Remington's Pharmaceutical Sciences (19th ed.) (Genarro, ed. (1995) Mack Publishing Co., Easton, Pa.). Preferably, pharmaceutical carriers are chosen based upon the intended mode of administration of a glycogenolysis inhibitor, optionally in combination with one or more other chemotherapeutic agents. The pharmaceutically acceptable carrier may include, for example, disintegrants, binders, lubricants, glidants, emollients, humectants, thickeners, silicones, flavoring agents, and water.

A glycogenolysis inhibitor, optionally in combination with one or more other chemotherapeutic agents, may be incorporated with excipients and administered in the form of ingestible tablets, buccal tablets, troches, capsules, elixirs, suspensions, syrups, wafers, or any other form known in the pharmaceutical arts. The pharmaceutical compositions may also be formulated in a sustained release form. Sustained release compositions, enteric coatings, and the like are known in the art. Alternatively, the compositions may be a quick release formulation.

As disclosed herein, an effective amount of a glycogenolysis inhibitor, optionally in combination with one or more other chemotherapeutic agents, in the described methods is an amount sufficient, when used as described herein, to inhibit or decrease glycogenolysis in the subject targeted for treatment when compared to the absence of the combination. An effective amount of a glycogenolysis inhibitor alone or in combination may vary based on a number of factors, including but not limited to, the activity of the active compounds, the physiological characteristics of the subject, the nature of the condition to be treated, and the route and/or method of administration. General dosage ranges of certain compounds are provided herein and in the cited references based on animal models of various types of cancer. Various conversion factors, formulas, and methods for determining human dose equivalents of animal dosages are known in the art, and are described, e.g., in Freireich et al., Cancer Chemother Repts 50(4): 219 (1966), Monro et al., Toxicology Pathology, 23: 187-98 (1995), Boxenbaum and Dilea, J. Clin. Pharmacol. 35: 957-966 (1995), and Voisin et al., Reg. Toxicol. Pharmacol., 12(2): 107-116 (1990), which are herein incorporated by reference.

The disclosed methods typically involve the administration of a glycogenolysis inhibitor, optionally in combination with one or more other chemotherapeutic agents, in a dosage range of from about 0.001 ng/kg/day to about 200 mg/kg/day. Other non-limiting dosages include from about 0.001 to about 0.01 ng/kg/day, about 0.01 to about 0.1 ng/kg/day, about 0.1 to about 1 ng/kg/day, about 1 to about 10 ng/kg/day, about 10 to about 100 ng/kg/day, about 100 ng/kg/day to about 1 .mu.g/kg/day, about 1 to about 2 .mu.g/kg/day, about 2 .mu.g/kg/day to about 0.02 mg/kg/day, about 0.02 to about 0.2 mg/kg/day, about 0.2 to about 2 mg/kg/day, about 2 to about 20 mg/kg/day, or about 20 to about 200 mg/kg/day. However, as understood by those skilled in the art, the exact dosage of a glycogenolysis inhibitor, optionally in combination with one or more other chemotherapeutic agents, used to treat a particular condition will vary in practice due to a wide variety of factors. Accordingly, dosage guidelines provided herein are not limiting as the range of actual dosages, but rather provide guidance to skilled practitioners in selecting dosages useful in the empirical determination of dosages for individual patients. Advantageously, methods described herein allow treatment of one or more conditions with reductions in side effects, dosage levels, dosage frequency, treatment duration, safety, tolerability, and/or other factors. So where suitable dosages for a glycogenolysis inhibitor to modulate glycogenolysis activity are known to a skilled person, the disclosure includes the use of about 75%, about 50%, about 33%, about 25%, about 20%, about 15%, about 10%, about 5%, about 2.5%, about 1%, about 0.5%, about 0.25%, about 0.2%, about 0.1%, about 0.05%, about 0.025%, about 0.02%, about 0.01%, or less than the known dosage.

In some embodiments, an effective, chemotherapeutic amount is an amount that achieves a concentration within the target tissue, using the particular mode of administration, at or above the IC50 for activity of a glycogenolysis inhibitor. In some embodiments, the glycogenolysis inhibitor is administered in a manner and dosage that gives a peak concentration of about 1, 1.5, 2, 2.5, 5, 10, 20 or more times the IC50 concentration. IC50 values and bioavailability data for various glycogenolysis inhibitors are known in the art, and are described, e.g., in the references cited herein.

In further embodiments, an effective modulating amount is a dose that lies within a range of circulating concentrations that includes the ED50 (the pharmacologically effective dose in 50% of subjects) with little or no toxicity.

In some embodiments, an effective glycogenolysis inhibiting amount is an amount that achieves a peak concentration within the target tissue, using the particular mode of administration, at or above the IC50 or EC50 concentration for the modulation of cancer. In various embodiments, a glycogenolysis inhibitor is administered in a manner and dosage that gives a peak concentration of about 1, 1.5, 2, 2.5, 5, 10, 20 or more times the IC50 or EC50 concentration for the modulation of glycogenolysis. In some embodiments, the IC50 or EC50 concentration for the modulation of neurogenesis is substantially lower than the IC50 concentration for activity of a GABA agent, allowing treatment of conditions for which it is beneficial to modulate glycogenolysis with lower dosage levels, dosage frequencies, and/or treatment durations relative to known therapies.

In other embodiments, the amount of a glycogenolysis inhibitor used in vivo may be about 50%, about 45%, about 40%, about 35%, about 30%, about 25%, about 20%, about 18%, about 16%, about 14%, about 12%, about 10%, about 8%, about 6%, about 4%, about 2%, or about 1% or less than the maximum tolerated dose for a subject, including where one or more other chemotherapeutic agents is used in combination with the glycogenolysis inhibitor. This is readily determined for each glycogenolysis inhibitor that has been in clinical use or testing, such as in humans.

Alternatively, the amount of a glycogenolysis inhibitor, optionally in combination with one or more other chemotherapeutic agents, may be an amount selected to be effective to produce an improvement in a treated subject based on detectable glycogenolysis in vitro as described above. In some embodiments, such as in the case of a known glycogenolysis inhibitor, the amount is one that minimizes clinical side effects seen with administration of the agent to a subject. The amount of an agent used in vivo may be about 50%, about 45%, about 40%, about 35%, about 30%, about 25%, about 20%, about 18%, about 16%, about 14%, about 12%, about 10%, about 8%, about 6%, about 4%, about 2%, or about 1% or less of the maximum tolerated dose in terms of acceptable side effects for a subject. This is readily determined for each glycogenolysis inhibitor or other agent(s) of a combination disclosed herein as well as those that have been in clinical use or testing, such as in humans.

In some methods described herein, the application of a glycogenolysis inhibitor in combination with one or more other chemotherapeutic agents may allow effective treatment with substantially fewer and/or less severe side effects compared to existing treatments. In some embodiments, combination therapy with a glycogenolysis inhibitor and one or more additional chemotherapeutic agents allows the combination to be administered at dosages that would be sub-therapeutic when administered individually or when compared to other treatments. In some cases, methods described herein allow treatment of certain conditions for which treatment with the same or similar compounds is ineffective using known methods due, for example, to dose-limiting side effects, toxicity, and/or other factors.

In other embodiments, each agent in a combination of agents may be present in an amount that results in fewer and/or less severe side effects than that which occurs with a larger amount. Thus the combined effect of the glycogenolysis inhibitor will provide a desired chemotherapeutic activity while exhibiting fewer and/or less severe side effects overall.

The pharmaceutical composition may be formulated by one having ordinary skill in the art with compositions selected depending upon the chosen mode of administration. Suitable pharmaceutical carriers are described in the most recent edition of Remington's Pharmaceutical Sciences, A. Osol, a standard reference text in this field.

Administering the pharmaceutical composition can be effected or performed using any of the various methods known to those skilled in the art. Systemic formulations include those designed for administration by injection, e.g. subcutaneous, intravenous, intramuscular, intrathecal or intraperitoneal injection, as well as those designed for transdermal, transmucosal, oral or pulmonary administration.

For injection, the compounds of the invention may be formulated in aqueous solutions, preferably in physiologically compatible buffers such as Hanks's solution, Ringer's solution, or physiological saline buffer. The solution may contain formulatory agents such as suspending, stabilizing and/or dispersing agents. Injectables are sterile and pyrogen free. Alternatively, the compounds may be in powder form for constitution with a suitable vehicle, e.g., sterile pyrogen-free water, before use. For transmucosal administration, penetrants appropriate to the barrier to be permeated are used in the formulation. Such penetrants are generally known in the art.

For parenteral administration, the glycogenolysis inhibitor can be, for example, formulated as a solution, suspension, emulsion or lyophilized powder in association with a pharmaceutically acceptable parenteral vehicle. Examples of such vehicles are water, saline, Ringer's solution, dextrose solution, 5% human serum albumin, Ringer's dextrose, dextrose and sodium chloride, lactated Ringer's and fixed oils, polyethylene glycol, polyvinyl pyrrolidone, lecithin, arachis oil or sesame oil. Liposomes and nonaqueous vehicles such as fixed oils may also be used. The vehicle or lyophilized powder may contain additives that maintain isotonicity (e.g., sodium chloride, mannitol) and chemical stability (e.g., buffers and preservatives). The formulation is sterilized by commonly used techniques. Parenteral dosage forms may be prepared using water or another sterile carrier. For example, a parenteral composition suitable for administration by injection is prepared by dissolving 1.5% by weight of active ingredient in 0.9% sodium chloride solution. Alternatively, the solution can be lyophilised and then reconstituted with a suitable solvent just prior to administration.

Pharmaceutically acceptable carriers are well known to those skilled in the art and include, but are not limited to, 0.01-0.1 M and preferably 0.05 M phosphate buffer or 0.8% saline. Intravenous carriers include fluid and nutrient replenishers, electrolyte replenishers such as those based on Ringer's dextrose, and the like. Additionally, such pharmaceutically acceptable carriers can be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, ethanol, alcoholic/aqueous solutions, glycerol, emulsions or suspensions, including saline and buffered media.

The pharmaceutical compositions can be prepared using conventional pharmaceutical excipients and compounding techniques. Oral dosage forms may be elixers, syrups, tablets, pills, dragees, capsules, liquids, gels, syrups, slurries, suspensions and the like, for oral ingestion by a patient to be treated. The typical solid carrier may be an inert substance such as lactose, starch, glucose, cellulose preparations such as maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP); granulating agents; binding agents, magnesium sterate, dicalcium phosphate, mannitol and the like. A composition in the form of a capsule can be prepared using routine encapsulation procedures. For example, pellets containing the active ingredient can be prepared using standard carrier and then filled into a hard gelatin capsule; alternatively, a dispersion or suspension can be prepared using any suitable pharmaceutical carrier(s), for example, aqueous gums, celluloses, silicates or oils and the dispersion or suspension then filled into a soft gelatin capsule. Typical liquid oral excipients include ethanol, glycerol, glycerine, non-aqueous solvent, for example, polyethylene glycol, oils, or water with a suspending agent, preservative, flavoring or coloring agent and the like. All excipients may be mixed as needed with disintegrants, diluents, lubricants, and the like using conventional techniques known to those skilled in the art of preparing dosage forms. If desired, disintegrating agents may be added, such as the cross-linked polyvinylpyrrolidone, agar, or alginic acid or a salt thereof such as sodium alginate. If desired, solid dosage forms may be sugar-coated or enteric-coated using standard techniques. For oral liquid preparations such as, for example, suspensions, elixirs and solutions, suitable carriers, excipients or diluents include water, glycols, oils, alcohols, etc. Additionally, flavoring agents, preservatives, coloring agents and the like may be added.

For buccal administration, the compounds may take the form of tablets, lozenges, and the like formulated in conventional manner. The compounds may also be formulated in rectal or vaginal compositions such as suppositories or enemas. A typical suppository formulation comprises a binding and/or lubricating agent such as polymeric glycols, glycerides, gelatins or cocoa butter or other low melting vegetable or synthetic waxes or fats. For administration by inhalation, the compounds for use according to the present invention are conveniently delivered in the form of an aerosol spray from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The formulations may also be a depot preparation which can be administered by implantation (for example subcutaneously or intramuscularly) or by intramuscular injection. In such embodiments, the compounds may be formulated with suitable polymeric or hydrophobic materials (for example as an emulsion in an acceptable oil) or ion exchange resins, or as sparingly soluble derivatives, for example, as a sparingly soluble salt.

Alternatively, other pharmaceutical delivery systems may be employed. Liposomes and emulsions are well known examples of delivery vehicles that may be used. Certain organic solvents such as dimethylsulfoxide also may be employed, although usually at the cost of greater toxicity. Additionally, the compounds may be delivered using a sustained-release system, such as semipermeable matrices of solid polymers containing the therapeutic agent. Various of sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The compounds used in the invention may also be formulated for parenteral administration by bolus injection or continuous infusion and may be presented in unit dose form, for instance as ampoules, vials, small volume infusions or pre-filled syringes, or in multi-dose containers with an added preservative.

Preservatives and other additives can also be present, such as, for example, antimicrobials, antioxidants, chelating agents, inert gases and the like. All carriers can be mixed as needed with disintegrants, diluents, granulating agents, lubricants, binders and the like using conventional techniques known in the art.

Routes of Administration

As described, the methods of the disclosure comprise contacting a cell with a glycogenolysis inhibitor, optionally in combination with one or more other chemotherapeutic agents, or administering such an agent or combination to a subject, to result in treatment of cancer. Some embodiments comprise the use of one glycogenolysis inhibitor in combination with one or more other chemotherapeutic agents. In other embodiments, a combination of two or more of the above agents, is used in combination with one or more other chemotherapeutic agents.

In some embodiments, methods of treatment disclosed herein comprise the step of administering to a mammal a glycogenolysis inhibitor, optionally in combination with one or more other chemotherapeutic agents, for a time and at a concentration sufficient to treat the condition targeted for treatment. The disclosed methods can be applied to individuals having, or who are likely to develop, disorders relating to cancer.

Depending on the desired clinical result, the disclosed agents or pharmaceutical compositions are administered by any means suitable for achieving a desired effect. Various delivery methods are known in the art and can be used to deliver an agent to a subject or to cells within a tissue of interest, such as a tumor. The delivery method will depend on factors such as the tissue of interest, the nature of the compound, and the duration of the experiment or treatment, among other factors. For example, an osmotic minipump can be implanted into a tumor. Alternatively, compounds can be administered by direct injection into the tumor. Compounds can also be administered into the periphery (such as by intravenous or subcutaneous injection, or oral delivery).

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1

### Glycogen Metabolism in Clear Cell RCC

Methods

From analysis of microarray and enzymatic data, it can be hypothesized that gluconeogenesis and mitochondrial function is reduced, and glycolysis is increased in RCC. These factors make RCC particularly reliant on glucose, which is obtained externally via glucose transporters or internally from glycogen stores. In order to further define the metabolism of RCC human RCC biopsies were analyzed, as well as normal grossly uninvolved kidney tissue as a control. The samples (n=4) were homogenized, normalized for total protein content, applied to SDS-PAGE and the resulting immunoblots stained with antibodies of regulatory metabolic proteins.

Findings

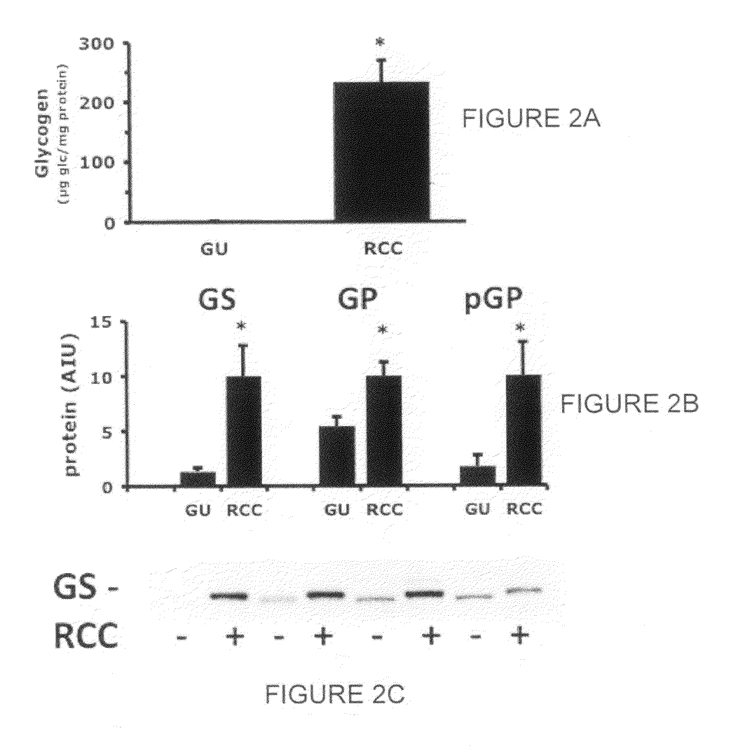

As expected, grossly uninvolved kidney tissue contained very little glycogen at 1.3±0.6 μg glucose/mg protein, whereas RCC contained about 180-fold more glycogen at 230±40 μg glucose/mg protein (FIG. 2A). These values fall within the ranges reported elsewhere (Steinberg, P et al. (1992); Mayer, D. and P. Bannasch (1988)). Glycogen synthase protein levels are increased 8 fold (p<0.05; FIG. 2B). The enzyme is apparently also inhibited, as indicated by the phosphorylation-dependent gel shift (FIG. 2C). Phosphorylated glycogen synthase requires higher concentrations of glucose-6-phosphate required to activate the enzyme (Kaslow, H. R. et al. J. Biol. Chem. 254(11):4674-7 (1979)). This is supported by a report detailing a two-fold increase in glucose-6-phosphate in RCC (Mayer, D. and P. Bannasch (1988)). Glycogen phosphorylase levels also increase about 85% (p<0.05; FIG. 2B), which is consistent with reported values (Takashi, M. et al. (1989)). Phosphorylation of glycogen phosphorylase activates the enzyme and staining with a phospho-specific glycogen phosphorylase antibody showed a 5.8-fold increase in RCC (p<0.05; FIG. 2B) (Fischer, E. H. and E. G. Krebs J. Biol. Chem. 216:121-32 (1955)). This implies that the enzyme is activated and that glycogen metabolism is integrated into carcinoma metabolism.

TABLE 1
mRNA and Protein Levels of Metabolic Proteins

| | Microarray Data* | | | Proteomic |
|---|---|---|---|---|
| | S 1‡ | S 4 | | Data** |
| Up-Regulated Proteins | | | | |
| (RCC/Normal Kidney) | | | | |
| GLUT1 | 1.5 | 3.7 | | |
| hexokinase | 1.4 | 0.9 | | 1.6^{e} |
| glycogen synthase | 1.4 | 1.2 | | 8.9^{f} |
| glycogen phosphoylase | | | | 3.1^{f}, 2.1^{g} |
| phosphofructokinase | 3.4 | 7.8 | | |
| enolase | 1.1 | 2.9 | 26.2^{a}, 6.2^{b} | |
| phosphotriose isomerase | 1.6 | 3.3 | 1.4^{c} | |
| phosphoglycerate dH | 1.1 | 2.5 | | 1.7^{e}, 5.5^{h} |
| phosphoglycerate kinase | 2.4 | 4.7 | | 3.2^{d} |
| pyruvate kinase | | | 1.3^{c} | 4.3^{e} |
| lactate dH | 2.2 | 4.2 | 1.3^{c} | 5.4^{d} |
| β-actin | 1.6 | 1.7 | | |
| MnSOD | 1.6 | 5.1 | | 5.4^{d} |
| Down-Regulated Proteins | | | | |
| (RCC/Normal Kidney) | | | | |
| L-phosphoglycerate dH | 4.1 | 5.6 | 5.6^{a} | |
| L-pyruvate kinase | 1.1 | 1.5 | | |
| L-aldolase B | 70.9 | 22.9 | 8.3^{a}, 13.6^{b} | |
| L-glucose-6-phophatase | | | 9.3^{a} | 5.0^{e} |
| L-fructose bisphosphate | 4.3 | 4.4 | 8.4^{a}, 4.7^{b} | 4.9^{e} |
| Phosphoenolpyruvate | 6.2 | 11.8 | 10.9^{a}, 8.9^{b} | |
| Isocitrate dH | 1.4 | 1.7 | 1.8^{c} | |
| Succinate dH | 1.5 | 1.2 | | |
| Complex I | 1.3 | 2.1 | 4.9^{b}, 1.6^{c} | |
| Complex III | | | 1.5^{c} | |
| Complex IV | | | 2.0^{c} | |
| ATP synthase (Complex V) | | | 2.0^{c} | 1.7^{h} |

‡Stage 1 and 4 RCC tumor cDNA levels taken from Boer et al.
*Microarray data taken from ^{a}Liou et al., ^{b}Takashi et al., and ^{C}Young, et al.
**Proteomic data taken from ^{d}Shi et al., enzyme activity data taken from ^{e}Steinberg et al., ^{f}Mayer et al., ^{g}Takashi et al., and ^{H}Cuezva et al.

Mitochondrial Metabolism in RCC

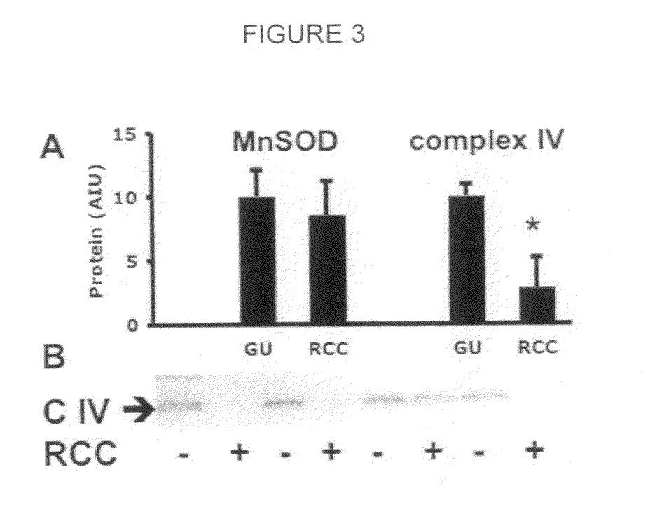

The shift to glycolytic metabolism reduces the reliance on oxygen delivery and mitochondrial metabolism allowing RCC cells to survive in an oxygen poor environment (Gatenby, R. A. and R. J. Gillies Nat. Rev. Cancer 4(11):891-9 (2004)). Levels of manganese superoxide dismutase (MnSOD), a matrix protein and indicator of mitochondrial mass, and subunit 1 of complex IV (cytochrome oxidase) of the electron transport chain (FIG. 3) were measured. No change in MnSOD levels between grossly uninvolved and RCC tissue were found, indicating that mitochondrial mass is unchanged, although there are reports of specific up-regulation of MnSOD in RCC and other cancers (Shi, T. et al. Mol. Carcinog. 40(1):47-61 (2004); Hussain, S. P. et al. Cancer Res. 64(7):2350-6 (2004)). Levels of subunit 1 of complex IV were also examined, which is considered a regulatory point in the electron transport chain. There was a 72% reduction in complex IV levels in the RCC tissue (p<0.05; FIG. 3). The removal of any component of the electron transport chain can lead to an elimination of oxidative phosphorylation. Others have found that subunit 1 of complex V, ATP synthase, is also reduced in RCC (Cuezva, J. M. et al. Cancer Res. 62(22):6674-81 (2002)). These observations show that oxidative phosphorylation is probably reduced in RCC.

Energy Homeostasis in RCC

The inventors hypothesize that the accumulated glycogen in RCC is dynamic and necessary for cellular metabolism. It provides an abundant supply of glucose to an RCC cell that is highly reliant on glucose metabolism. RCC can be very good at trapping external glucose, in spite of apparently lower levels of glucose transporters, and there can still be some residual flux through the gluconeogenic pathway. Inhibition of glucose release from glycogen can also deprive the RCC cell of a resource on which it is particularly dependent. This can cause a reduction in tumor growth and increase susceptibility to other treatments, especially those that compromise nutrient delivery, such as the anti-angiogenic VEGF-receptor inhibitor SU-11248.

Measurement of mRNA and Protein Levels of Regulatory Enzymes in Glucose Metabolism.

The microarray data outlined above (Table 1) details a phenotypic shift away from oxidative metabolism and towards increased glucose uptake and glycolysis. This is consistent with expected changes in carcinoma metabolism. Enzymes involved in glucose oxidation can be decreased and enzymes involved in anaerobic glycolysis can be increased. Mechanisms that trap glucose in the RCC cell can be up regulated and the gluconeogenic pathway reduced but not eliminated.

Measurement of mRNA and Protein Levels of Transcriptional Regulators of Glucose Homeostasis.

To examine the underlying transcriptional mechanisms, mRNA and protein levels of oncogenes and metabolic regulators can be measured (Akt, H-ras, LKB1, and AMPK) (Elstrom, R. L. et al. Cancer Res. 64(11):3892-9 (2004), Shaw, R. J. et al. Science 310(5754):1642-6 (2005)). Upstream transcription factors and transcriptional co-activators that are known to regulate whole metabolic pathways are also tested (c-myc, ARα, PGC-1α, PGC-1β, TORC2, SIRT1) (Osthus, R. C. et al. J. Biol. Chem. 275(29):21797-800 (2000); Xu, J. et al. J. Biol. Chem. 277(52):50237-44 (2002), Mootha, V. K. et al. Proc. Natl. Acad. Sci. USA 101(17):6570-5 (2004), Rodgers, J. T. et al. Nature 434(7029):113-8 (2005), Koo, S. H. et al. Nature 437(7062):1109-11 (2005)). Hypoxia inducible factors-1 and ±2 (HIF-1 and HIF-2) which are up regulated in a large proportion of RCC as a result of VHL down regulation or ablation (Gnarra J. R. et al. Nat. Genet. 7(1):85-90 (1994); Raval, R. R. et al. Mol. Cell. Biol. 25(13):5675-86 (2005), Sufan, R. I. et al. Am. J. Physiol. Renal Physiol. 287(1):F1-6 (2004) Herman, J. G. et al. Proc. Natl. Acad. Sci. USA 91(21):9700-4 (1994), Krieg, M. Oncogene 19(48):5435-43 (2000).

The patterns of transcriptional control can reflect increased glycolysis, decreased oxidative metabolism and decreased but not eliminated gluconeogenesis. Activation of transcriptional regulation can be uneven. This will provide clues about specific transcriptional mechanisms linking RCC gene expression the glycogen-rich phenotype.

### Example 2

### Use of Glycogenolysis Inhibitors in Mouse Model of Renal Cell Carcinoma

Measurement of the Effect of Glycogenolysis Inhibitors on a Mouse Model of Rcc.

The inventors hypothesize that inhibiting the release of glucose from internal glycogen stores restricts RCC tumor growth and is synergistic with other treatments, in particular those that compromise external access to glucose, such as anti-angiogenic compounds. To test this Renca RCC cells would be injected orthotopically into a mouse kidney and then treated with combinations of the following compounds:
CP-316819, a glycogen phosphorylase inhibitor (Ercan-Fang, N. et al. (2005)).
NN-nonyl-deoxynojirimycin (NN-DNJ), an inhibitor of lysosomal glycogenolysis and glycogen debranching enzyme (Andersson, U. et al. (2004).
IL-2, the current standard of care in human RCC (Murphy, W. J. et al. J. Immunol. 170(5):2727-33 (2003)).
SU-11248 (sunitinib, Sutent), a VEGF-receptor tyrosine kinase inhibitor (Murray, L. J. et al. Clin. Exp. Metastasis 20(8):757-66 (2003)).

TABLE 3
Treatment pattern of RCC cohorts

| Cohort | 1 | 2 | 3 | 4 | 5 | 6 | 7 | 8 | 9 |
|---|---|---|---|---|---|---|---|---|---|
| CP-316819 | − | − | − | + | + | + | − | − | − |
| NN-DNJ | − | − | − | − | − | − | + | + | + |
| IL-2 | − | + | − | − | + | − | − | + | − |
| SU-11248 | − | − | + | − | − | + | − | − | + |

This is a total of nine treatment cohorts (see Table 3). The growth of the tumor in situ is determined by conducting microCT scans and obtaining volume measurements of both the tumor bearing kidney and control contralateral kidney. The affected kidney would be allowed to grow to over 10 times its original volume before the animal is euthanized under a given set of physiological criteria (Nanus, D. M. and Engelstein (2001), Nishisaka, N. et al. (2001)). This allows for the measurement of both the rate of increase of the tumor index (ratio of Renca-treated kidney volume over the control kidney volume) as well as the survival period before euthanasia (Nanus, D. M. and Engelstein (2001), Nishisaka, N. et al. (2001) Gruys, M. E. et al. Cancer Res. 61(16):6255-63 (2001)).

Both of the glycogenolysis inhibitors can reduce the rate of Renca tumor growth and prolong the survival period before euthanasia. Of the two inhibitors, α-glucosidase inhibitor (NN-DNJ) inhibits both lysosomal and cytosolic glycogenolysis. The glycogenolysis inhibitors can be synergistic with the anti-angiogenic SU-11248. This combination compromises accessibility to both external glucose uptake (reduced angiogenesis) and internal glycogen reserves. Co-treatment of either CP-316819 or NN-DNJ with IL-2 could also be synergistic since the effects of IL-2 may induce the RCC cells to rely more on glycogen reserves.

Methods

The Renca mouse model is generated by orthotopic injection of 5×10⁵ Renca cells into the left kidney of BALB/c mice. After 14 days chronically catheterize the animal into the jugular. This gives the animal time to recover and pretreatments to begin. At day 28, the Renca tumor should be between 7 to 8 cm³ (Nanus, D. M. and Engelstein (2001), Nishisaka, N. et al. (2001)). Described above, with the following exceptions: no catheterization will take place and only 10⁵ Renca cells are injected in to the kidney. The size of the affected kidney is measured in situ using the General Electric Medical Systems EVS-RS9 scanner in the University of Utah small animal imaging facility and rendered as a three dimensional figure by the Scientific and Computing Imaging Institute at the University of Utah. The tumor index is calculated as the ratio of affected over unaffected kidney volumes. Euthanasia is done under a fixed set of criteria, including hematuria, obvious discomfort or moribund behavior (reduced food and water intake and reduced mobility). A successful outcome is a significant (p<0.05) reduction in tumor growth or longer survival periods. Based on the observed growth rate variations for RCC-derived cell lines published in the literature, and using a two-tailed T-test and a power of 0.9, a total of 20 mice in each cohort can be used to detect a 25% decrease in growth rate of the tumor (p<0.05) (Gruys, M. E. et al. (2001) (Gruys, M. E. et al. (2001)).

Following injection of Renca cells, mice are daily dosed orally with vehicle containing 10% DMSO/0.1% Pluronic P105 Block Copolymer Surfactant (BASF, Parsippany, N.J.) for 4 days, followed on the fifth day with vehicle containing 25 mg CP-316819/kg or vehicle alone (Ercan-Fang, N. et al. (2005)). The mice are then treated for 5 days out of every seven for the course of the study. CP-316819 is a gift from Pfizer Inc. N-nonyl-deoxynijiromycin (NN-DNJ): RCC-treated BALB/c mice are treated with vehicle alone or with 50 mg NN-DNJ/kg/day for 21 days (Andersson, U. et al. (2004)). Mice are dosed orally through gavage. NN-DNJ is commercially available (Toronto Research Chemicals, North York, ON). Interleukin-2 (IL-2): Mice are treated twice daily with 300 000 IU IL-2 i.p. (Chiron Corp., Emeryville, Calif.), twice a week for a total of eight injections (Murphy, W. J. et al. (2003)). SU-11248 (sunitinib, Sutent): Mice are injected intraperitoneally with 40 mg/kg/day for 21 days (Murray, L. J. et al. (2003), Schueneman, A. J. et al. Cancer Res. 63(14):4009-16 (2003)).

### Example 3

### In Vitro Model of Renal Cell Carcinoma

The metabolism of carcinoma is distinctive in that it does not depend on the availability of oxygen. This restricts the metabolic options for the carcinoma and results in an increased dependence on anaerobic glucose metabolism. Many carcinoma types, particularly renal cell carcinoma, endometrial and thyroid carcinoma, also have the super accumulations of glycogen, which is the mechanism by which cells store glucose reserves. Given the increased metabolic demands of the carcinoma, these accumulations can be hypothesized to be beneficial to carcinoma cell survival. Restricting metabolic access to these reserves can be hypothesized to increase the susceptibility of carcinoma to chemotherapies, particularly anti-angiogenic agents that restrict external nutrient availability.

Establishment of an In Vitro Model of RCC.

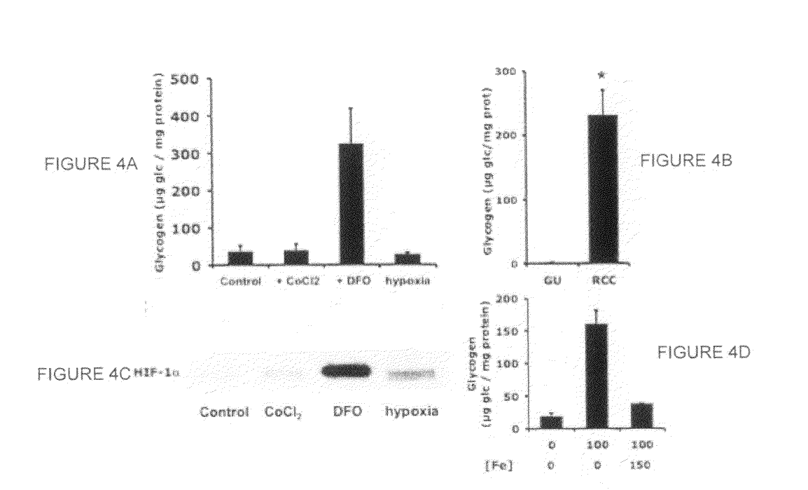

To test this hypothesis it was first necessary to establish an in vitro model of glycogenosis in a relevant clear cell renal cell carcinoma (ccRCC) cell type (CAKI-2 cells). It was first necessary to induce levels of glycogenosis similar to those measured in human disease. We obtained human RCC biopsies (FIG. 4 B) and compared glycogen levels with neighboring grossly uninvolved tissue (GU). There was a 180-fold induction of glycogen levels in human RCC (1±1 to 230±40 μg glucose/mg protein, p<0.01). We treated human Caki-2 RCC cells with known activators of hypoxia pathway in 20 mM glucose for 24 h: 100 μM cobalt chloride, 100 μM desferrioxamine (DFO), and hypoxia (pO₂=1%) (FIG. 4 A). Only DFO, the iron chelator, was able to induce glycogenosis to a level equivalent of that observed in human RCC biopsies (320±90 μg glucose/mg protein, p<0.01). Likewise, only DFO was able to stabilize HIF protein levels, implying, but not proving, a casual relationship (FIG. 4 C). DFO-dependent accumulation did not occur when incubated in 150 μM ferric ammonium citrate (FIG. 4 D). Iron depletion was significantly more effective at inducing HIF protein levels compared to other activators, strongly implying that iron is a limiting factor in ccRCC metabolism and glycogenosis.

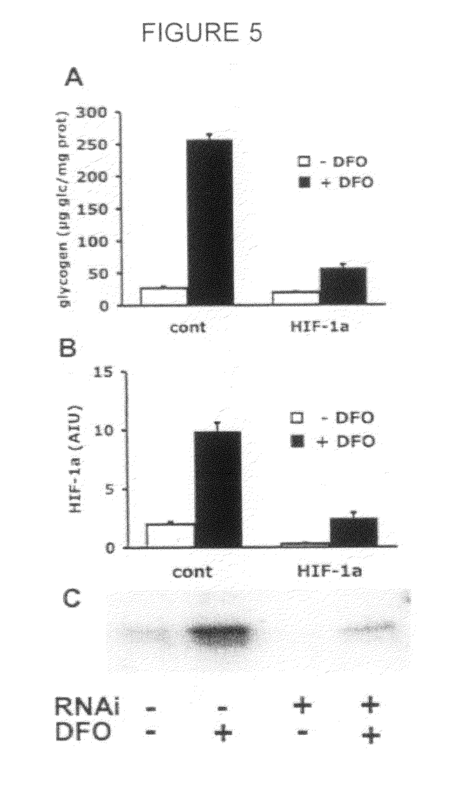

While DFO induced both HIF-1α and glycogenosis, it remained to be established that glycogenosis was HIF-1α dependent. We therefore repeated the induction of glycogenosis with DFO after prior treatment with HIF-1α RNAi (FIG. 5). Glycogen levels increased 9-fold in CAKI-2 cells with DFO treatment (p<0.05). Specific suppression of 1-HIF-1α protein levels, confirmed by immunoblot (B,C), resulted in a suppression of DFO-induced glycogenosis (A) (p<0.05), establishing that DFO-induced glycogenosis is HIF-1α dependent.

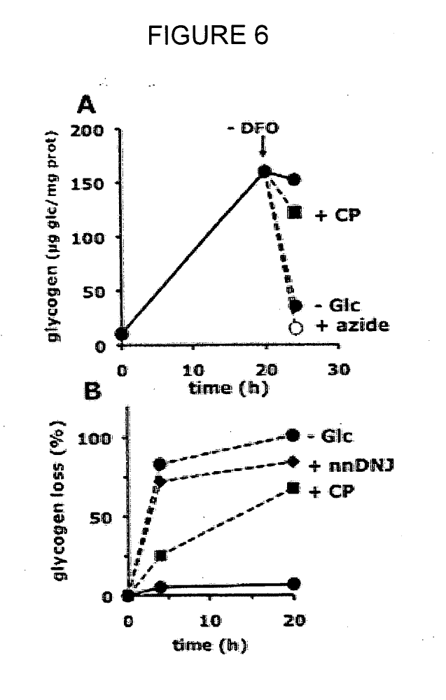

To demonstrate that glycogen metabolism was integrated to ccRCC metabolism CAKI-2 cells, glycogenosis was induced by DFO-treatment and then exposed to different metabolic states (FIG. 6A). Caki-2 cells loose glycogen when starved of glucose in the media and not when there was abundant glucose (p<0.05). As proposed in the invention glycogen reserves are responsive to the metabolic status of the carcinoma cell and are mobilized in response to metabolic stress.

We also tested whether glycogen mobilization could be inhibited pharmacologically (FIG. 6B). We therefore treated glucose-starved CAKI-2 cells with two types of glycogenolysis inhibitor: CP-316819 (▪), which inhibits glycogen phosphorylase and NN-DNJ (♦), which inhibits lysosomal glycogen breakdown. The glycogen phosphorylase inhibitor (FIG. 6B, +CP) was most effective at reducing glycogen breakdown. After 4 h glycogen loss was restricted to 25% and roughly ⅓^{rd} was still present after 20 h in glucose free media (p<0.05). This indicates that most glycogen was mobilized through glycogen phosphorylase. This demonstrates that glycogen mobilization can be specifically targeted.

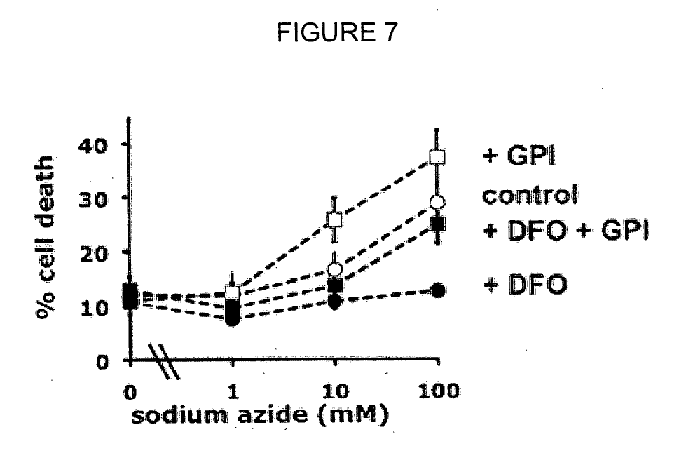

This invention has been experimentally verified in an in vitro model of renal cell carcinoma (FIG. 7). When these cells are subjected to an environment of restricted nutrient availability, they begin to undergo cell death. As predicted, prior accumulation of glycogen in these cells results in protection against cell death (solid line). However, this protection is lost when co-treated with an agent that prevents glycogen breakdown, (CP-316819 (Pfizer, Inc.), closed circles).

The invention was tested in an in vitro model. CAKI-2 cells were pre-incubated in the presence of 300 μM desferrioximine (DFO, solid line) in RPMI1640 and 10% fetal calf serum to induce the accumulation of glycogen. After 16 hours the media was changed to DMEM with 0 mM glucose and 0 mM pyruvate for 4 hours, to mimic the absence of nutrient as present in the interior of a tumor or resulting from treatment with anti-angiogenic inhibitors, increasing concentrations of sodium azide (0 to 100 mM) to mimic the absence of oxygen, and the presence or absence of 60 μM CP-316819 to specifically target glycogenolysis (CP, closed circles). Cells were then trypsinized, collected, treated with 0.1 mg propidium iodide in KRBH buffer (25 mM HEPES 7.4, 120 mM NaCl, 4.6 mM KCl, 1.9 mM CaCl2, 1 mM MgSO4, and 1.2 mM KH2PO4), and analyzed for cell death by measuring inclusion of propidium iodide on a FACScan instrument (Beckman-Coulter). Cells pretreated with glycogen accumulation were significantly resistant to cell death in 100 mM azide (*, solid line, open symbols, p<0.001). Treatment with the glycogenolysis inhibitor CP-316819 (solid line, closed symbols) removed that protection, demonstrating that the invention is experimentally verified in an in vitro model of RCC.

### Example 4

### Use of Iron Treatment to Reduce Glycogenosis

As outlined in Example 2 and FIGS. 4 to 7, iron levels are potentially limiting to RCC cells. Depletion of iron with DFO treatment results in replication of glycogenosis as observed in human renal cancer (FIG. 4A) and induction of HIF-1α protein levels in the human CAKI-2 renal cancer cell line. The addition of excess iron prevents DFO-induced glycogenosis, demonstrating that the effect is dependent on low iron concentrations.

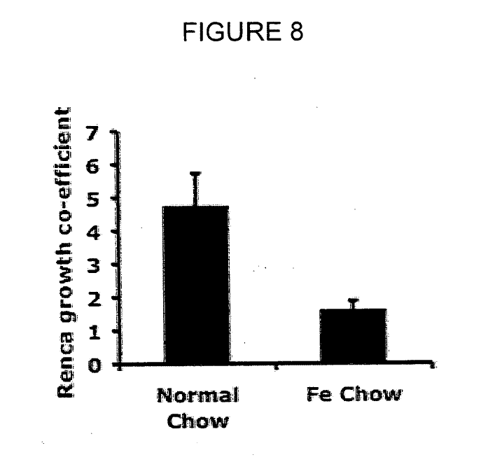

Just as inhibition of glycogenolysis can remove the protection afforded by ccRCC glycogenosis, prevention of glycogenosis by iron treatment could also have the same effect. Mice were fed with or without an iron-rich diet for 6 to 8 weeks. They were then injected with Renca cells as described in Example 2. Mice pre-treated with an iron-rich diet had reduced Renca growth rates, growing 3-fold slower (FIG. 8, p=0.05, n=3). These results show that the invention has the potential to reduce tumor growth rate by reduction of glycogen levels.

### Example 5

### Use of Glycogenolysis Inhibitors to Increase Sensitivity of Tumor Detection with Positron-Emission Tomography (PET)

As outlined in FIG. 1, glucose is available from either intracellular glycogen, or extracellular blood glucose. The metabolic changes with carcinogenesis result in increased dependence on the extracellular delivery of glucose. This has been exploited to detect tumors using a positron-emitting glucose derivative and Positron-Emission Tomography (PET). RCC however is often not detectible using this methodology. We propose that this insensitivity is due to the glycogenosis present in RCC, providing an alternate source of glucose. Pre-treatment with a glycogenolysis inhibitor will increase the sensitivity of PET because RCC would then be required to import glucose to meet the metabolic demands of the carcinoma cells. As glycogen is often coupled to glycolysis we propose that other carcinomas will be able to be detected with increased sensitivity.

### REFERENCES

American Cancer Society, Cancer Reference Information. www.acs.org, 2005.
Motzer, R. J. and P. Russo, Systemic therapy for renal cell carcinoma. J. Urol., 2000, 163(2):408-17.
Vogelzang, N. J. and W. M. Stadler, Kidney cancer. Lancet, 1998, 352(9141):1691-6.
Kim, H. L., D. Seligson, X. Liu, N. Janzen, M. H. Bui, H. Yu, T. Shi, A. S. Belldegrun, S. Horvath, and R. A. FIG. 1 in, Using tumor markers to predict the survival of patients with metastatic renal cell carcinoma. J. Urol., 2005, 173(5):1496-501.
Drucker, B. J., Renal cell carcinoma: current status and future prospects. Cancer Treat. Rev., 2005, 31(7):536-45.
Steinberg, P., S. Storkel, F. Oesch, and W. Thoenes, Carbohydrate metabolism in human renal clear cell carcinomas. Lab. Invest., 1992, 67(4):506-11.
Mulders, P., R. FIG. 1 in, J. B. deKernion, R. Wiltrout, M. Linehan, D. Parkinson, W. deWolf, and A. Belldegrun, Renal cell carcinoma: recent progress and future directions. Cancer Res., 1997, 57(22):5189-95.
Mayer, D. and P. Bannasch, Activity of glycogen synthase and phosphorylase and glucose 6-phosphate content in renal clear cell carcinomas. J. Cancer Res. Clin. Oncol., 1988, 114(4):369-72.
Liou, L. S., T. Shi, Z. H. Duan, P. Sadhukhan, S. D. Der, A. A. Novick, J. Hissong, M. Skacel, A. Almasan, and J. A. DiDonato, Microarray gene expression profiling and analysis in renal cell carcinoma. BMC Urol., 2004, 4:9.
Takahashi, M., D. R. Rhodes, K. A. Furge, H. Kanayama, S. Kagawa, B. B. Haab, and B. T. Teh, Gene expression profiling of clear cell renal cell carcinoma: gene identification and prognostic classification. Proc. Natl. Acad. Sci. USA, 2001, 98(17):9754-9.
Higgins, J. P., R. Shinghal, H. Gill, J. H. Reese, M. Terris, R. J. Cohen, M. Fero, J. R. Pollack, M. van de Rijn, and J. D. Brooks, Gene expression patterns in renal cell carcinoma assessed by complementary DNA microarray. Am. J. Pathol., 2003, 162(3):925-32.
Boer, J. M., W. K. Huber, H. Sultmann, F. Wilmer, A. von Heydebreck, S. Haas, B. Korn, B. Gunawan, A. Vente, L. Fuzesi, M. Vingron, and A. Poustka, Identification and classification of differentially expressed genes in renal cell carcinoma by expression profiling on a global human 31,500-element cDNA array. Genome Res., 2001, 11(11):1861-70.
Kardas, I., K. Mrozek, M. Babinska, K. Krajka, P. Hadaczek, J. Lubinski, A. Roszkiewicz, E. Kuziemska, and J. Limon, Cytogenetic and molecular findings in 75 clear cell renal cell carcinomas. Oncol. Rep., 2005, 13(5):949-56.
Sugimura, J., G. Tamura, Y. Suzuki, and T. Fujioka, Allelic loss on chromosomes 3p, 5q and 17p in renal cell carcinomas. Pathol. Int., 1997, 47(2-3):79-83.
Alimov, A., B. Sundelin, N. Wang, C. Larsson, and U. Bergerheim, Loss of 14q31-q32.2 in renal cell carcinoma is associated with high malignancy grade and poor survival. Int. J. Oncol., 2004, 25(1):179-85.
Hirata, H., H. Matsuyama, H. Matsumoto, Y. Korenaga, C. Ohmi, S. Sakano, S. Yoshihiro, and K. Naito, Deletion mapping of 18q in conventional renal cell carcinoma. Cancer Genet. Cytogenet., 2005, 163(2):101-5.
Gnarra, J. R., K. Tory, Y. Weng, L. Schmidt, M. H. Wei, H. Li, F. Latif, S. Liu, F. Chen, F. M. Duh, and et al., Mutations of the VHL tumour suppressor gene in renal carcinoma. Nat. Genet., 1994, 7(1):85-90.
van Houwelingen, K. P., B. A. van Dijk, C. A. Hulsbergen-van de Kaa, L. J. Schouten, H. J. Gorissen, J. A. Schalken, P. A. van den Brandt, and E. Oosterwijk, Prevalence of von Hippel-Lindau gene mutations in sporadic renal cell carcinoma: results from The Netherlands cohort study. BMC Cancer, 2005, 5(1):57.
George, D. J. and W. G. Kaelin, Jr., The von Hippel-Lindau protein, vascular endothelial growth factor, and kidney cancer. N. Engl. J. Med., 2003, 349(5):419-21.
Maynard, M. A. and M. Ohh, von Hippel-Lindau Tumor Suppressor Protein and Hypoxia-Inducible Factor in Kidney Cancer. Am. J. Nephrol., 2004, 24:1-13.
Raval, R. R., K. W. Lau, M. G. Tran, H. M. Sowter, S. J. Mandriota, J. L. Li, C. W. Pugh, P. H. Maxwell, A. L. Harris, and P. J. Ratcliffe, Contrasting properties of hypoxia-inducible factor 1 (HIF-1) and HIF-2 in von Hippel-Lindau-associated renal cell carcinoma. Mol. Cell. Biol., 2005, 25(13):5675-86.
Griffin, J. L. and J. P. Shockcor, Metabolic profiles of cancer cells. Nat. Rev. Cancer, 2004, 4(7):551-61.
Hockel, M. and P. Vaupel, Tumor Hypoxia: Definitions and Current Clinical, Biologic, and Molecular Aspects. Journal of the National Cancer Institute, 2001, 93(4):266-76.
Gatenby, R. A. and R. J. Gillies, Why do cancers have high aerobic glycolysis? Nat. Rev. Cancer, 2004, 4(11):891-9.
Maxwell, P. H., C. W. Pugh, and P. J. Ratcliffe, Activation of the HIF pathway in cancer. Current Opinion in Genetics and Development, 2001, 11:293-9.
Sufan, R. I., M. A. Jewett, and M. Ohh, The role of von Hippel-Lindau tumor suppressor protein and hypoxia in renal clear cell carcinoma. Am. J. Physiol. Renal Physiol., 2004, 287(1):F1-6.
Semenza, G. L., Targeting HIF-1 for cancer therapy. Nat Rev Cancer, 2003. 3(10): p. 721-32.
Wagstaff, P., H. Y. Kang, D. Mylott, P. J. Robbins, and M. K. White, Characterization of the avian GLUT1 glucose transporter: differential regulation of GLUT1 and GLUT3 in chicken embryo fibroblasts. Mol. Biol. Cell., 1995, 6(11):1575-89.
Kozma, L., K. Baltensperger, J. Klarlund, A. Porras, E. Santos, and M. P. Czech, The ras signaling pathway mimics insulin action on glucose transporter translocation. Proc. Natl. Acad. Sci. USA, 1993, 90(10):4460-4.
Osthus, R. C., H. Shim, S. Kim, Q. Li, R. Reddy, M. Mukherjee, Y. Xu, D. Wonsey, L. A. Lee, and C. V. Dang, Deregulation of Glucose Transporter 1 and Glycolytic Gene Expression by c-Myc. J. Biol. Chem., 2000, 275(29):21797-800.
Elstrom, R. L., D. E. Bauer, M. Buzzai, R. Karnauskas, M. H. Harris, D. R. Plas, H. Zhuang, R. M. Cinalli, A. Alavi, C. M. Rudin, and C. B. Thompson, Akt stimulates aerobic glycolysis in cancer cells. Cancer Res., 2004, 64(11):3892-9.
Krown, S. E., Interferon treatment of renal cell carcinoma. Current status and future prospects. Cancer, 1987, 59(3 Suppl):647-51.
Siegel, J. P. and R. K. Puri, Interleukin-2 toxicity. J. Clin. Oncol., 1991, 9(4):694-704.
Yang, J. C., L. Haworth, R. M. Sherry, P. Hwu, D. J. Schwartzentruber, S. L. Topalian, S. M. Steinberg, H. X. Chen, and S. A. Rosenberg, A randomized trial of bevacizumab, an anti-vascular endothelial growth factor antibody, for metastatic renal cancer. N Engl. J. Med., 2003, 349(5):427-34.
Motzer, R. J., M. Dror Michaelson, B. G. Redman, G. R. Hudes, G. Wilding, R. A. FIG. 1 in, M. S. Ginsberg, S. T. Kim, C. M. Baum, S. E. Deprimo, J. Z. Li, C. L. Bello, C. P. Theuer, D. J. George, and B. I. Rini, Activity of SU11248, a Multitargeted Inhibitor of Vascular Endothelial Growth Factor Receptor and Platelet-Derived Growth Factor Receptor, in Patients With Metastatic Renal Cell Carcinoma. J. Clin. Oncol., 2006, 24(1):16-24.
Faivre, S., C. Delbaldo, K. Vera, C. Robert, S. Lozahic, N. Lassau, C. Bello, S. Deprimo, N. Brega, G. Massimini, J. P. Armand, P. Scigalla, and E. Raymond, Safety, Pharmacokinetic, and Antitumor Activity of SU11248, a Novel Oral Multitarget Tyrosine Kinase Inhibitor, in Patients With Cancer. J. Clin. Oncol., 2005.
Young, A. N., M. B. Amin, C. S. Moreno, S. D. Lim, C. Cohen, J. A. Petros, F. F. Marshall, and A. S. Neish, Expression profiling of renal epithelial neoplasms: a method for tumor classification and discovery of diagnostic molecular markers. Am. J. Pathol., 2001, 158(5):1639-51.
Shi, T., F. Dong, L. S. Liou, Z. H. Duan, A. C. Novick, and J. A. DiDonato, Differential protein profiling in renal-cell carcinoma. Mol. Carcinog., 2004, 40(1):47-61.
Cuezva, J. M., M. Krajewska, M. L. de Heredia, S. Krajewski, G. Santamaria, H. Kim, J. M. Zapata, H. Marusawa, M. Chamorro, and J. C. Reed, The bioenergetic signature of cancer: a marker of tumor progression. Cancer Res., 2002, 62(22):6674-81.
Warburg, O., On the origin of cancer cells. Science, 1956, 123(3191):309-14.
Nielsen, J. N., J. F. Wojtaszewski, R. G. Haller, D. G. Hardie, B. E. Kemp, E. A. Richter, and J. Vissing, Role of 5′AMP-activated protein kinase in glycogen synthase activity and glucose utilization: insights from patients with McArdle's disease. J. Physiol., 2002, 541(Pt 3):979-89.
Takashi, M., T. Koshikawa, N. Kurobe, and K. Kato, Elevated concentrations of brain-type glycogen phosphorylase in renal cell carcinoma. Jpn. J. Cancer Res., 1989, 80(10):975-80.
Roach, P. J., Glycogen and its metabolism. Curr. Mol. Med., 2002, 2(2):101-20.
Arad, M., B. J. Maron, J. M. Gorham, W. H. Johnson, Jr., J. P. Saul, A. R. Perez-Atayde, P. Spirito, G. B. Wright, R. J. Kanter, C. E. Seidman, and J. G. Seidman, Glycogen storage diseases presenting as hypertrophic cardiomyopathy. N Engl. J. Med., 2005, 352(4):362-72.
Hoover, D. J., S. Lefkowitz-Snow, J. L. Burgess-Henry, W. H. Martin, S. J. Armento, I. A. Stock, R. K. McPherson, P. E. Genereux, E. M. Gibbs, and J. L. Treadway, Indole-2-carboxamide inhibitors of human liver glycogen phosphorylase. J. Med. Chem., 1998, 41(16):2934-8.
Lee, W. N., P. Guo, S. Lim, S. Bassilian, S. T. Lee, J. Boren, M. Cascante, V. L. Go, and L. G. Boros, Metabolic sensitivity of pancreatic tumour cell apoptosis to glycogen phosphorylase inhibitor treatment. Br. J. Cancer, 2004, 91(12):2094-100.
Schnier, J. B., K. Nishi, P. H. Gumerlock, F. A. Gorin, and E. M. Bradbury, Glycogen synthesis correlates with androgen-dependent growth arrest in prostate cancer. BMC Urol., 2005, 5(1):6.
Ercan-Fang, N., M. R. Taylor, J. L. Treadway, C. B. Levy, P. E. Genereux, E. M. Gibbs, V. L. Rath, Y. Kwon, M. C. Gannon, and F. Q. Nuttall, Endogenous effectors of human liver glycogen phosphorylase modulate effects of indole-site inhibitors. Am. J. Physiol. Endocrinol. Metab., 2005, 289(3):E366-72.
Andersson, U., G. Reinkensmeier, T. D. Butters, R. A. Dwek, and F. M. Platt, Inhibition of glycogen breakdown by imino sugars in vitro and in vivo. Biochem. Pharmacol., 2004, 67(4):697-705.
Bollen, M. and W. Stalmans, The antiglycogenolytic action of 1-deoxynojirimycin results from a specific inhibition of the alpha-1,6-glucosidase activity of the debranching enzyme. Eur. J. Biochem., 1989, 181(3):775-80.
Mellor, H. R., D. C. Neville, D. J. Harvey, F. M. Platt, R. A. Dwek, and T. D. Butters, Cellular effects of deoxynojirimycin analogues: uptake, retention and inhibition of glycosphingolipid biosynthesis. Biochem. J., 2004, 381(Pt 3):861-6.
Branza-Nichita, N., D. Durantel, S. Carrouee-Durantel, R. A. Dwek, and N. Zitzmann, Antiviral effect of N-butyldeoxynojirimycin against bovine viral diarrhea virus correlates with misfolding of E2 envelope proteins and impairment of their association into E1-E2 heterodimers. J. Virol., 2001, 75(8):3527-36.
Kaslow, H. R., R. D. Eichner, and S. E. Mayer, Interconversion between multiple glucose 6-phosphate-dependent forms of glycogen synthase in intact adipose tissue. J. Biol. Chem., 1979, 254(11):4674-7.
Fischer, E. H. and E. G. Krebs, Conversion of Phosphorylase b to Phosphorylase a in Muscle Extracts. J. Biol. Chem., 1955, 216:121-32.
Ahn, Y. S., H. Zerban, R. Grobholz, and P. Bannasch, Sequential changes in glycogen content, expression of glucose transporters and enzymic patterns during development of clear/acidophilic cell tumors in rat kidney. Carcinogenesis, 1992, 13(12):2329-34.
Majhail, N. S., J. L. Urbain, J. M. Albani, M. H. Kanvinde, T. W. Rice, A. C. Novick, T. M. Mekhail, T. E. Olencki, P. Elson, and R. M. Bukowski, F-18 fluorodeoxyglucose positron emission tomography in the evaluation of distant metastases from renal cell carcinoma. J. Clin. Oncol., 2003, 21(21):3995-4000.
Kang, D. E., R. L. White, Jr., J. H. Zuger, H. C. Sasser, and C. M. Teigland, Clinical use of fluorodeoxyglucose F 18 positron emission tomography for detection of renal cell carcinoma. J. Urol., 2004, 171(5):1806-9.
Hussain, S. P., P. Amstad, P. He, A. Robles, S. Lupold, I. Kaneko, M. Ichimiya, S. Sengupta, L. Mechanic, S. Okamura, L. J. Hofseth, M. Moake, M. Nagashima, K. S. Forrester, and C. C. Harris, p53-induced up-regulation of MnSOD and GPx but not catalase increases oxidative stress and apoptosis. Cancer Res., 2004, 64(7):2350-6.
Shaw, R. J., K. A. Lamia, D. Vasquez, S. H. Koo, N. Bardeesy, R. A. Depinho, M. Montminy, and L. C. Cantley, The kinase LKB1 mediates glucose homeostasis in liver and therapeutic effects of metformin. Science, 2005, 310(5754):1642-6.
Xu, J., G. Xiao, C. Trujillo, V. Chang, L. Blanco, S. B. Joseph, S. Bassilian, M. F. Saad, P. Tontonoz, W. N. Lee, and I. J. Kurland, Peroxisome proliferator-activated receptor alpha (PPARalpha) influences substrate utilization for hepatic glucose production. J. Biol. Chem., 2002, 277(52):50237-44.
Mootha, V. K., C. Handschin, D. Arlow, X. Xie, J. St Pierre, S. Sihag, W. Yang, D. Altshuler, P. Puigserver, N. Patterson, P. J. Willy, I. G. Schulman, R. A. Heyman, E. S. Lander, and B. M. Spiegelman, Erralpha and Gabpa/b specify PGC-1alpha-dependent oxidative phosphorylation gene expression that is altered in diabetic muscle. Proc. Natl. Acad. Sci. USA, 2004, 101(17):6570-5.
Rodgers, J. T., C. Lerin, W. Haas, S. P. Gygi, B. M. Spiegelman, and P. Puigserver, Nutrient control of glucose homeostasis through a complex of PGC-1alpha and SIRT1. Nature, 2005, 434(7029):113-8.
Koo, S. H., L. Flechner, L. Qi, X. Zhang, R. A. Screaton, S. Jeffries, S. Hedrick, W. Xu, F. Boussouar, P. Brindle, H. Takemori, and M. Montminy, The CREB coactivator TORC2 is a key regulator of fasting glucose metabolism. Nature, 2005, 437(7062):1109-11.
Herman, J. G., F. Latif, Y. Weng, M. I. Lerman, B. Zbar, S. Liu, D. Samid, D. S. Duan, J. R. Gnarra, W. M. Linehan, and et al., Silencing of the VHL tumor-suppressor gene by DNA methylation in renal carcinoma. Proc. Natl. Acad. Sci. USA, 1994, 91(21):9700-4.
Krieg, M., R. Haas, H. Brauch, T. Acker, I. Flume, and K. H. Plate, Up-regulation of hypoxia-inducible factors HIF-1alpha and HIF-2alpha under normoxic conditions in renal carcinoma cells by von Hippel-Lindau tumor suppressor gene loss of function. Oncogene, 2000, 19(48):5435-43.
Nanus, D. M. and D. Engelstein, Orthotopic Model of Renal Cell Carcinoma, in Renal Cancer Methods and Protocols, J. H. Mydlo, Editor, 2001, Humana Press Inc.: Totowa, N.J. p. 245-53.

Nishisaka, N., P. Morse, R. F. Jones, C. Y. Wang, and G. P. Haas, Murine Animal Model, in Renal Cancer: Methods and Protocols, J. H. Mydlo, Editor, 2001, Humana Press Inc.: Totowa, N.J. p. 255-64.
Bandsma, R. H., A. Grefhorst, T. H. van Dijk, F. H. van der Sluijs, A. Hammer, D. J. Reijngoud, and F. Kuipers, Enhanced glucose cycling and suppressed de novo synthesis of glucose-6-phosphate result in a net unchanged hepatic glucose output in ob/ob mice. Diabetologia, 2004, 47(11):2022-31.
Ng, L.-K. and M. Hupe, Simple gas chromatographic method for the assay of salts of carboxylic acids as their trimethylsilyl derivatives. J. Chromatography, 1993, 637:104-8.
Murphy, W. J., L. Welniak, T. Back, J. Hixon, J. Subleski, N. Seki, J. M. Wigginton, S. E. Wilson, B. R. Blazar, A. M. Malyguine, T. J. Sayers, and R. H. Wiltrout, Synergistic anti-tumor responses after administration of agonistic antibodies to CD40 and IL-2: coordination of dendritic and CD8+ cell responses. J. Immunol., 2003, 170(5):2727-33.
Murray, L. J., T. J. Abrams, K. R. Long, T. J. Ngai, L. M. Olson, W. Hong, P. K. Keast, J. A. Brassard, A. M. O'Farrell, J. M. Chemington, and N. K. Pryer, SU11248 inhibits tumor growth and CSF-1R-dependent osteolysis in an experimental breast cancer bone metastasis model. Clin. Exp. Metastasis, 2003, 20(8):757-66.
Gruys, M. E., T. C. Back, J. Subleski, T. A. Wiltrout, J. K. Lee, L. Schmidt, M. Watanabe, R. Stanyon, J. M. Ward, J. M. Wigginton, and R. H. Wiltrout, Induction of transplantable mouse renal cell cancers by streptozotocin: in vivo growth, metastases, and angiogenic phenotype. Cancer Res., 2001, 61(16):6255-63.
Schueneman, A. J., E. Himmelfarb, L. Geng, J. Tan, E. Donnelly, D. Mendel, G. McMahon, and D. E. Hallahan, SU11248 maintenance therapy prevents tumor regrowth after fractionated irradiation of murine tumor models. Cancer Res., 2003, 63(14):4009-16.

What is claimed is:

1. A method of treating a subject with cancer, the method comprising: administering to the subject an effective amount of at least one agent that inhibits glycogenolysis in combination with at least one chemotherapeutic agent.

2. The method of claim 1, wherein the cancer is clear cell carcinoma.

3. The method of claim 2, wherein the clear cell carcinoma is renal cell carcinoma.

4. The method of claim 1, wherein the glycogenolysis inhibitor inhibits at least one of glycogen phosphorylase, lysosomal α-glucosidase and phosphoglucomutase.

5. The method of claim 3, wherein the glycogenolysis inhibitor is N-(n-Nonyl)deoxynojirimycin.

6. The method of claim 3, wherein the glycogenolysis inhibitor is CP-3616819.

7. The method of claim 1, wherein the chemotherapeutic agent is azide.

8. The method of claim 1, wherein the chemotherapeutic agent is an anti-angiogenic compound.

9. The method of claim 8, wherein the anti-angiogenic compound is a VEGF-receptor inhibitor.

10. The method of claim 9, wherein the VEGF-receptor inhibitor is SU-11248.

11. The method of claim 1, wherein the chemotherapeutic agent is at least one of interleukin-2 and interferon-α.

12. The method of claim 1, wherein the glycogenolysis inhibitor decreases cellular metabolism, thereby increasing effectiveness of the chemotherapeutic agent.

13. A method of treating an individual having clear cell carcinoma, the method comprising: administering to the individual an effective amount of at least one agent that inhibits glycogenolysis.

14. The method of claim 13, wherein the clear cell carcinoma is renal cell carcinoma.

15. The method of claim 13, wherein the glycogenolysis inhibitor inhibits at least one of glycogen phosphorylase, lysosomal α-glucosidase and phosphoglucomutase.

16. The method of claim 13, wherein the glycogenolysis inhibitor is N-(n-Nonyl)deoxynojirimycin.

17. The method of claim 13, wherein the glycogenolysis inhibitor is CP-3616819.

18. A method of diagnosing and monitoring renal cell carcinoma in an individual comprising: obtaining a sample from the individual; measuring in the sample a level of at least one protein involved in cellular metabolism; and determining a presence or an increased metastatic potential of clear cell carcinoma based on the level of the at least one protein involved in cellular metabolism.

19. The method of claim 16, wherein the at least one protein is an enzyme involved in anaerobic non-oxidative glucose metabolism.

20. The method of claim 17, wherein the enzyme involved in anaerobic non-oxidative glucose metabolism is up-regulated indicating the presence or the increased metastatic potential renal cell carcinoma.

21. The method of claim 16, wherein the at least one protein is an enzyme involved in oxidative metabolism.

22. The method of claim 19, wherein the enzyme involved in oxidative metabolism is down-regulated indicating the presence or increased metastatic potential of renal cell carcinoma.

23. A method of detecting cancer in a subject, the method comprising: administering to the subject an inhibitor of glycogenolysis; then administering to the subject a glucose-based composition; then monitoring uptake of the glucose-based composition by the cancer, thereby detecting the cancer.

24. The method of claim 23, wherein the cancer is clear cell carcinoma.

25. The method of claim 24, wherein the cancer is renal cancer.

26. The method of claim 23, wherein the glucose-based composition is fluorodeoxyglucose.

27. The method of claim 23, wherein administering an inhibitor of glycogenolysis results in a 20% or more increase uptake of the glucose-based composition by the cancer cells.

28. The method of claim 23, wherein the uptake of the glucose-based composition is monitored by a PET scan.
